(19) United States
(12) Patent Application Publication
(10) Pub. No.: US 2025/0248773 A1
(43) Pub. Date: Aug. 7, 2025

(54) SYSTEMS AND METHODS FOR PLANNING AND ASSISTING ORTHOPAEDIC SURGICAL PROCEDURES

(71) Applicant: DePuy Ireland Unlimited Company, Ringaskiddy (IE)

(72) Inventor: Daniel GIRARDEAU-MONTAUT, Grenoble (FR)

(21) Appl. No.: 19/037,120

(22) Filed: Jan. 25, 2025

Related U.S. Application Data
(60) Provisional application No. 63/550,849, filed on Feb. 7, 2024.

(51) Int. Cl. A61B 34/30; A61B 17/15; A61B 34/10; A61B 34/20; A61F 2/38; A61F 2/46

(52) U.S. Cl. CPC A61B 34/30; A61B 17/155; A61F 2/389; A61F 2/461; A61B 2034/107; A61B 2034/2068; A61F 2002/4633; A61F 2002/4658

(57) ABSTRACT

A method for an orthopaedic surgical procedure includes capturing, using a surgical navigation system, a plurality of measurements of a knee joint of a patient including measurements on an operative side of a femur of the patient relative to a femur coordinate space, measurements on an operative side of a tibia of the patient relative to a tibia coordinate space, and a spatial relationship between the femur coordinate space and the tibia coordinate space at each of a plurality of different poses of the knee joint. The method also includes developing, using the surgical navigation system, a surgical plan for the orthopaedic surgical procedure based on the plurality of measurements. The surgical plan includes a position of a tibial prosthesis in the tibia coordinate space, and a position of a femoral prosthesis in the femur coordinate space. A surgical navigation system is also disclosed.

This application claims priority under 35 U.S.C. § 119 to U.S. Patent Application Ser. No. 63/550,849, which was filed on Feb. 7, 2024 and is incorporated herein by reference in its entirety.

### TECHNICAL FIELD

The present disclosure relates generally to orthopaedic surgical procedures and, more particularly, to systems and methods for planning and assisting unicompartmental knee arthroplasty (UKA).

### BACKGROUND

Joint arthroplasty is a well-known surgical procedure by which a diseased and/or damaged natural joint is replaced by a prosthetic joint, which may include one or more orthopaedic implants. To facilitate replacement of a natural joint with a prosthetic joint, orthopaedic surgeons may use a variety of orthopaedic surgical instruments such as, for example, surgical saws, cutting guides, reamers, broaches, drill guides, drills, positioners, insertion tools and/or other surgical instruments. A surgeon may use manual instruments such as cutting blocks or other cutting guides to perform the various resections in an orthopaedic procedure. Alternatively, or in addition, a surgeon may use a computer-assisted surgical navigation system, such as a robotic-assisted surgical system, to perform the various resections in an orthopaedic procedure. For instance, a robotic-assisted surgical system may assist a surgeon in performing a UKA to replace one of the two compartments of a patient's knee with a prosthetic joint.

### SUMMARY

According to an aspect of the present disclosure, a method for an orthopaedic surgical procedure may include capturing, using a surgical navigation system, a plurality of measurements of a knee joint of a patient and developing, using the surgical navigation system, a surgical plan for the orthopaedic surgical procedure based on the plurality of measurements. The plurality of measurements may include (i) one or more measurements on an operative side of a femur of the patient relative to a femur coordinate space, (ii) one or more measurements on an operative side of a tibia of the patient relative to a tibia coordinate space, and (iii) a spatial relationship between the femur coordinate space and the tibia coordinate space at each of a plurality of different poses of the knee joint. The surgical plan may include (i) a position of a tibial prosthesis in the tibia coordinate space, including an internal-external rotation of the tibial prosthesis and (ii) a position of a femoral prosthesis in the femur coordinate space, including an internal-external rotation of the femoral prosthesis. In some embodiments, developing the surgical plan may include defining a transverse vector in the tibia coordinate space that is representative of the internal-external rotation of the tibial prosthesis; using, for each of the plurality of different poses of the knee joint, the corresponding spatial relationship between the femur coordinate space and the tibia coordinate space to project the transverse vector from the tibia coordinate space into the femur coordinate space; combining the plurality of projected vectors to determine a composite vector in a transverse plane in the femur coordinate space; and planning the internal-external rotation of the femoral prosthesis based on the composite vector.

Additionally, in some embodiments, the position of the tibial prosthesis, including the internal-external rotation of the tibial prosthesis used when defining the transverse vector, is a planned position of the tibial prosthesis defined in the surgical plan before performing transverse and sagittal resections of the tibia during the orthopaedic surgical procedure. Additionally, the planned position of the tibial prosthesis, including the internal-external rotation of the tibial prosthesis used when defining the transverse vector, may have been modified by a surgeon from a default position set by the surgical navigation system.

In some embodiments, the position of the tibial prosthesis, including the internal-external rotation of the tibial prosthesis used when defining the transverse vector, may be an actual position of the tibial prosthesis defined in the surgical plan after performing a transverse resection of the tibia and a sagittal resection of the tibia during the orthopaedic surgical procedure. In such embodiments, capturing the one or more measurements on the operative side of the tibia may include capturing at least one measurement on at least one of (i) a transverse resected surface created by the transverse resection of the tibia and (ii) a sagittal resected surface created by the sagittal resection of the tibia.

Additionally, in some embodiments, the transverse vector in the tibia coordinate space may be defined along a sagittal axis of the tibial prosthesis. In such embodiments, planning the internal-external rotation of the femoral prosthesis may include rotating the position of the femoral prosthesis in the femur coordinate space to align a sagittal axis of the femoral prosthesis with the composite vector. Additionally, in some embodiments, the transverse vector in the tibia coordinate space is defined along a frontal axis of the tibial prosthesis. In such embodiments, planning the internal-external rotation of the femoral prosthesis may include rotating the position of the femoral prosthesis in the femur coordinate space to align a frontal axis of the femoral prosthesis with the composite vector.

In some embodiments, combining the plurality of projected vectors may include computing an average vector from the plurality of projected vectors and projecting the average vector onto the transverse plane to produce the composite vector. Additionally, in some embodiments, The method of claim 1, projecting the transverse vector from the tibia coordinate space into the femur coordinate space may include projecting the transverse vector onto a transverse plane in the femur coordinate space. In such embodiments, combining the plurality of projected vectors may include computing an average vector from the plurality of projected vectors to produce the composite vector.

According to another aspect of the present disclosure, a method for an orthopaedic surgical procedure may include capturing, using a surgical navigation system, a plurality of measurements of a knee joint of a patient and developing, using the surgical navigation system, a surgical plan for the orthopaedic surgical procedure based on the plurality of measurements. The plurality of measurements may include (i) one or more measurements on an operative side of a femur of the patient relative to a femur coordinate space, (ii) one or more measurements on an operative side of a tibia of the patient relative to a tibia coordinate space, and (iii) a spatial relationship between the femur coordinate space and the tibia coordinate space at each of a plurality of different poses of the knee joint. The plan may include (i) a position of a tibial prosthesis in the tibia coordinate space and (ii) a position of a femoral prosthesis in the femur coordinate space, including a medial-lateral translation of the femoral prosthesis. In such embodiments, developing the surgical plan may include using, for each of the plurality of different poses of the knee joint, the corresponding spatial relationship between the femur coordinate space and the tibia coordinate space, together with the position of the femoral prosthesis in the femur coordinate space and the position of the tibial prosthesis in the tibia coordinate space, to predict a contact location between the femoral prosthesis and the tibial prosthesis in the femur coordinate space. Additionally, developing the surgical plan may include combining a medial-lateral aspect of each of the plurality of predicted contact locations to determine a composite location in the femur coordinate space and planning the medial-lateral translation of the femoral prosthesis based on the composite location.

In some embodiments, the position of the tibial prosthesis used to determine the plurality of predicted contact locations may be embodied as a planned position of the tibial prosthesis defined in the surgical plan before performing transverse and sagittal resections of the tibia during the orthopaedic surgical procedure. In such embodiments, the planned position of the tibial prosthesis used to determine the plurality of predicted contact locations may have been modified by a surgeon from a default position set by the surgical navigation system.

Additionally, in some embodiments, the position of the tibial prosthesis used to determine the plurality of predicted contact locations may be embodied as an actual position of the tibial prosthesis defined in the surgical plan after performing a transverse resection of the tibia and a sagittal resection of the tibia during the orthopaedic surgical procedure. In such embodiments, capturing the one or more measurements on the operative side of the tibia may include capturing at least one measurement on at least one of (i) a transverse resected surface created by the transverse resection of the tibia and (ii) a sagittal resected surface created by the sagittal resection of the tibia.

In some embodiments, combining the medial-lateral aspect of each of the plurality of predicted contact locations may include computing an average of the medial-lateral aspects to produce the composite location. Additionally, planning the medial-lateral translation may include centering a mediolateral dimension of the femoral prosthesis on the composite location.

Additionally, in some embodiments, the plurality of different poses of the knee joint may include a first pose in which the femur and the tibia are in full extension and a second pose in which the femur and the tibia are flexed at 90 degrees. In such embodiments, the plurality of different poses of the knee joint further may include additional poses in a range of motion between the first pose and the second pose. The additional poses may be spaced at regular intervals of flexion between the first pose and the second pose. Further, in some embodiments, developing the surgical plan further may include determining a plurality of planes in the femur coordinate space for a plurality of resections of the femur based on the surgical plan's position of the femoral prosthesis.

According to a further aspect of the present disclosure, a method an orthopaedic surgical procedure capturing, using a surgical navigation system, a plurality of measurements of a femur of a patient relative to a femur coordinate space and developing, using the surgical navigation system, a surgical plan for the orthopaedic surgical procedure based on the plurality of measurements. The plurality of measurements may include (i) a most posterior point on an operative side of the femur and (ii) an anterior sizing point on the operative side of the femur. The surgical plan may include (i) a plurality of planes in the femur coordinate space for a plurality of resections of the femur and (ii) a size of a femoral prosthesis to be implanted on resected surfaces created by the plurality of resections of the femur. Additionally, developing the surgical plan may include selecting the size of the femoral prosthesis from among a plurality of possible sizes for the femoral prosthesis based on the most posterior point and the anterior sizing point.

In some embodiments, capturing the anterior sizing point on the operative side of the femur may include placing the femur and a tibia of the patient into full extension and touching a tip of a pointer instrument of the surgical navigation system to a point on the operative side of the femur that is directly proximal of a most anterior point of an anticipated transverse resection to be performed on the tibia.

Additionally, in some embodiments, capturing the anterior sizing point on the operative side of the femur may include placing the femur and a tibia of the patient into full extension and touching a tip of a pointer instrument of the surgical navigation system to a point on the operative side of the femur that is directly proximal of a most anterior point of an actual transverse resection performed on the tibia.

In some embodiments, selecting the size of the femoral prosthesis may include calculating a transverse distance in the femur coordinate space between (i) the most posterior point on the operative side of the femur and (ii) the anterior sizing point on the operative side of the femur and selecting, from among the plurality of possible sizes for the femoral prosthesis, the possible size with the largest anteroposterior dimension that does not exceed the transverse distance by more than one-half of a resolution of the surgical navigation system. Additionally or alternatively, selecting the size of the femoral prosthesis may include aligning, in the femur coordinate space, a digital model of each of the plurality of possible sizes for the femoral prosthesis to the most posterior point on the operative side of the femur and selecting, from among the plurality of possible sizes for the femoral prosthesis, the possible size corresponding to the aligned digital model with a smallest distance between (i) the anterior sizing point on the operative side of the femur and (ii) a most anterior point of the aligned digital model.

Additionally, in some embodiments, the method may further include performing, using the surgical navigation system, the plurality of resections of the femur according to the surgical plan. For example, performing the plurality of resections of the femur according to the surgical plan may include operating a robotic assisted surgery device in communication with the surgical navigation system. In such embodiments, for each of the plurality of resections of the femur, the robotic assisted surgery device may constrain movement of a surgical saw blade to a corresponding plane of the plurality of planes of the surgical plan.

According to yet a further aspect of the present disclosure, a method for an orthopaedic surgical procedure may include capturing, using a surgical navigation system, a plurality of measurements of a knee joint of a patient and developing, using the surgical navigation system, a surgical plan for the orthopaedic surgical procedure based on the plurality of measurements. The plurality of measurements may include (i) one or more measurements on an operative side of a femur of the patient relative to a femur coordinate space, (ii) one or more measurements on an operative side of a tibia of the patient relative to a tibia coordinate space, and (iii) a spatial relationship between the femur coordinate space and the tibia coordinate space while a surgeon positions the femur and the tibia in full extension with a target hip-knee-ankle angle. Additionally, the surgical plan may include (i) a transverse plane in the tibia coordinate space for a transverse resection of the tibia, (ii) a vertical dimension of a tibial prosthesis to be implanted on a transverse resected surface created by the transverse resection of the tibia, (iii) a distal plane in the femur coordinate space for a distal resection of the femur, and (iv) a vertical dimension of a femoral prosthesis to be implanted on a distal resected surface created by the distal resection of the femur. In such embodiments, developing the surgical plan may include calculating a first gap distance between the femoral prosthesis and the tibial prosthesis based on the transverse plane in the tibia coordinate space, the vertical dimension of the tibial prosthesis, the distal plane in the femur coordinate space, the vertical dimension of the femoral prosthesis, and the spatial relationship between the femur coordinate space and the tibia coordinate space. Additionally, developing the surgical plan may include, in response to the first gap distance being greater than one-half of a resolution of the surgical navigation system, updating the surgical plan by shifting the transverse plane proximally in the tibia coordinate space by the lesser of (i) the first gap distance and (ii) a current depth of the transverse resection of the tibia minus a minimum allowable value for the transverse resection of the tibia.

In some embodiments, developing the surgical plan may further include, after updating the surgical plan by shifting the transverse plane proximally in the tibia coordinate space, calculating a second gap distance between the femoral prosthesis and the tibial prosthesis based on the transverse plane in the tibia coordinate space, the vertical dimension of the tibial prosthesis, the distal plane in the femur coordinate space, the vertical dimension of the femoral prosthesis, and the spatial relationship between the femur coordinate space and the tibia coordinate space. Additionally, developing the surgical plan may further include, in response to the second gap distance being greater than one-half of a resolution of the surgical navigation system, updating the surgical plan by shifting the distal plane distally in the femur coordinate space by the lesser of (i) the second gap distance and (ii) a current depth of the distal resection of the femur minus a minimum allowable value for the distal resection of the femur.

Additionally, in some embodiments, developing the surgical plan further may include, after updating the surgical plan by shifting the distal plane distally in the femur coordinate space, calculating a third gap distance between the femoral prosthesis and the tibial prosthesis based on the transverse plane in the tibia coordinate space, the vertical dimension of the tibial prosthesis, the distal plane in the femur coordinate space, the vertical dimension of the femoral prosthesis, and the spatial relationship between the femur coordinate space and the tibia coordinate space. Additionally, developing the surgical plan further may include, in response to the third gap distance being greater than one-half of a resolution of the surgical navigation system, updating the surgical plan by selecting a new tibial prosthesis with a larger vertical dimension that minimizes that third gap distance.

In some embodiments, the tibial prosthesis may include a tibial tray and a bearing insert. In such embodiments, selecting the new tibial prosthesis with the larger vertical dimension may include selecting a new bearing insert for use with the tibial tray. Additionally, in some embodiments, the distal plane may be parallel to the transverse plane when the femur coordinate space and the tibia coordinate space have the spatial relationship.

The method may further include performing, using the surgical navigation system, the transverse resection of the tibia and the distal resection of the femur according to the surgical plan. In such embodiments, performing the transverse resection of the tibia and the distal resection of the femur according to the surgical plan may include operating a robotic assisted surgery device in communication with the surgical navigation system. The robotic assisted surgery device may constrain movement of a surgical saw blade to the transverse plane during the transverse resection of the tibia. Additionally, the robotic assisted surgery device may constrain movement of the surgical saw blade to the distal plane during the distal resection of the femur.

According to yet another aspect of the present disclosure, a method for an orthopaedic surgical procedure may include capturing, using a surgical navigation system, a plurality of measurements of a tibia of a patient relative to a tibia coordinate space and developing, using the surgical navigation system, a surgical plan for the orthopaedic surgical procedure based on the plurality of measurements. The plurality of measurements may include (i) a most collateral point on an operative side of the tibia and (ii) a tibial compartment border on the operative side of the tibia. The surgical plan may include (i) a plane in the tibia coordinate space for a transverse resection of the tibia and (ii) a size of a tibial tray of a tibial prosthesis to be implanted on a transverse resected surface created by the transverse resection of the tibia. Additionally, developing the surgical plan may include selecting the size of the tibial tray from among a plurality of possible sizes for the tibial tray based on the most collateral point and the tibial compartment border.

In some embodiments, capturing the tibial compartment border on the operative side of the tibia may include aligning an elongated body of a pointer instrument of the surgical navigation system with the tibial compartment border, prior to performing the transverse resection of the tibia. For example, in such embodiments, capturing the most collateral point on the operative side of the tibia may include touching a tip of the pointer instrument to a point in an anticipated plane of the transverse resection of the tibia, prior to performing the transverse resection.

Additionally, in some embodiments, capturing the tibial compartment border on the operative side of the tibia may include moving a tip of a pointer instrument of the surgical navigation system along a sagittal resected surface created by a sagittal resection of the tibia, after performing the transverse and sagittal resections of the tibia. In such embodiments, capturing the most collateral point on the operative side of the tibia may include touching the tip of the pointer instrument to a point in an actual plane of the transverse resection of the tibia, after performing the transverse resection of the tibia.

In some embodiments, selecting the size of the tibial tray may include calculating a smallest distance in the tibia coordinate space between (i) the most collateral point on the operative side of the tibia and (ii) the tibial compartment border on the operative side of the tibia and selecting, from among the plurality of possible sizes for the tibial tray, the possible size with the largest mediolateral dimension that does not exceed the smallest distance by more than one-half of a resolution of the surgical navigation system. Additionally or alternatively, selecting the size of the tibial tray may include aligning, in the tibia coordinate space, a digital model of each of the plurality of possible sizes for the tibial tray to the tibial compartment border on the operative side of the tibia and selecting, from among the plurality of possible sizes for the tibial tray, the possible size corresponding to the aligned digital model with a smallest distance between (i) the most collateral point on the operative side of the tibia and (ii) a most collateral point of the aligned digital model.

Additionally, in some embodiments, the method may further include performing, using the surgical navigation system, the transverse resection of the tibia according to the surgical plan. In such embodiments, performing the transverse resection of the tibia according to the surgical plan may include operating a robotic assisted surgery device in communication with the surgical navigation system. The robotic assisted surgery device may constrain movement of a surgical saw blade to the surgical plan's plane for the transverse resection of the tibia.

### BRIEF DESCRIPTION OF THE DRAWINGS

The concepts described herein are illustrated by way of example and not by way of limitation in the accompanying figures. For simplicity and clarity of illustration, elements illustrated in the figures are not necessarily drawn to scale. Where considered appropriate, reference labels have been repeated among the figures to indicate corresponding or analogous elements. The detailed description particularly refers to the accompanying figures in which:

FIG. 1 is a schematic diagram of a system for planning and assisting an orthopaedic surgical procedure;

FIG. 2 is a flow chart for an illustrative orthopaedic surgical procedure that may be performed with the system of FIG. 1;

FIGS. 3 and 4 are perspective diagrams of a femur and a tibia of a patient with surgical instruments during the orthopaedic surgical procedure of FIG. 2;

FIG. 5 is a perspective diagram of the tibia of the patient with a surgical instrument during the orthopaedic surgical procedure of FIG. 2;

FIG. 6 is a schematic diagram illustrating a display interface of the system of FIG. 1 during the orthopaedic surgical procedure of FIG. 2;

FIG. 7 is a perspective diagram illustrating tibial resections performed during the orthopaedic surgical procedure of FIG. 2;

FIG. 8 is a perspective diagram illustrating a posterior femoral resection performed during the orthopaedic surgical procedure of FIG. 2;

FIG. 9 is a perspective diagram illustrating a distal femoral resection performed during the orthopaedic surgical procedure of FIG. 2;

FIG. 10 is a perspective diagram illustrating a posterior chamfer femoral resection performed during the orthopaedic surgical procedure of FIG. 2;

FIG. 11 is a perspective diagram illustrating measurement of the patient's resected femur performed during the orthopaedic surgical procedure of FIG. 2;

FIG. 12 is a perspective diagram illustrating measurement of the patient's resected tibia performed during the orthopaedic surgical procedure of FIG. 2;

FIGS. 13A and 13B are flow charts for two illustrative methods of planning a size of a tibial prosthesis, which may be performed during the orthopaedic surgical procedure of FIG. 2;

FIGS. 14A and 14B are flow charts for two illustrative methods of planning a size of a femoral prosthesis, which may be performed during the orthopaedic surgical procedure of FIG. 2;

FIG. 15 is a flow chart for an illustrative method of planning a position of a femoral prosthesis to minimize edge loading, which may be performed during the orthopaedic surgical procedure of FIG. 2;

FIG. 16 is a flow chart for an illustrative method of planning a position of a femoral prosthesis to minimize overhang, which may be performed during the orthopaedic surgical procedure of FIG. 2; and

FIG. 17 is a flow chart for an illustrative method of developing a surgical plan to achieve a target hip-knee-ankle (HKA) angle for the patient, which may be performed during the orthopaedic surgical procedure of FIG. 2.

### DETAILED DESCRIPTION OF THE DRAWINGS

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

Terms representing anatomical references, such as anterior, posterior, medial, lateral, superior, inferior, etcetera, may be used throughout the specification in reference to the orthopaedic implants or prostheses and surgical instruments described herein as well as in reference to the patient's natural anatomy. Such terms have well-understood meanings in both the study of anatomy and the field of orthopaedics. Use of such anatomical reference terms in the written description and claims is intended to be consistent with their well-understood meanings unless noted otherwise.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

The disclosed embodiments may be implemented, in some cases, in hardware, firmware, software, or any combination thereof. The disclosed embodiments may also be implemented as instructions carried by or stored on a transitory or non-transitory machine-readable (e.g., computer-readable) storage medium, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

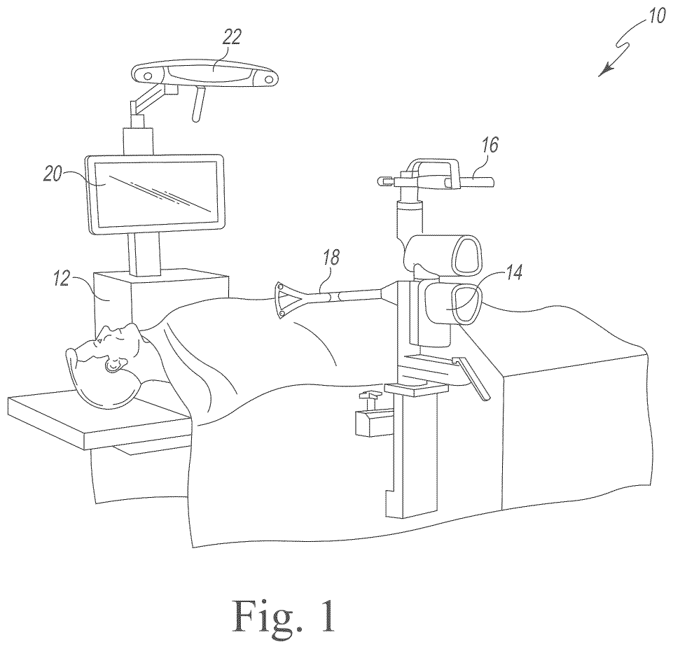

Referring now to FIG. 1, a surgical navigation system 10 is used during an orthopaedic surgical procedure, illustratively a unicompartmental knee arthroplasty (UKA). During that procedure, an orthopaedic surgeon uses the system 10 to capture measurements of various features of a patient's knee joint, to develop a surgical plan based on those measurements, and to perform resections of a femur and a tibia of the patient's knee joint according to the surgical plan. (As used in the present disclosure, “based on” does not exclude the presence of additional inputs, e.g., the surgeon and the system 10 may use additional information, beyond the captured measurements of the patient's knee joint, to develop the surgical plan.) As described in further detail below, the presently disclosed system 10 and methods may allow the surgeon to achieve a number of surgical objectives, including, but not limited to, more accurately reproducing the natural shape of the patient's femoral condyle being resurfaced, centering a femoral prosthesis over a tibial prosthesis to reduce or eliminate overhang, minimizing edge loading of the femoral and tibial prostheses throughout a range of motion of the patient's knee, and more closely achieving the surgeon's target hip-knee-ankle (HKA) angle for the patient.

As shown in FIG. 1, the system 10 includes a surgical planning and assistance device 12, a robotic assisted surgery device 14, and a number of tracking markers 18. The surgical planning and assistance device 12 may be embodied as any type of computer system capable of performing the functions described herein. For example, the surgical planning and assistance device 12 may be embodied as, without limitation, a workstation, a desktop computer, a laptop computer, a special-purpose compute device, a server, a rack-mounted server, a blade server, a network appliance, a web appliance, a tablet computer, a smartphone, a consumer electronic device, a distributed computing system, a multiprocessor system, and/or any other computing device capable of performing the functions described herein. Accordingly, the illustrative surgical planning and assistance device 12 may include components commonly found in a computer such as a processor, an I/O subsystem, memory, a data storage device, a communication subsystem, and various input/output devices. Additionally, although the surgical planning and assistance device 12 is illustrated in FIG. 1 as embodied as a single computer, it should be appreciated that the surgical planning and assistance device 12 may be embodied as multiple devices cooperating together to facilitate the functionality described below. For example, in some embodiments, the system 10 may include a base station and a satellite station or other combination of computing devices. Additionally or alternatively, in some embodiments, the surgical planning and assistance device 12 may be embodied as a “virtual server” formed from multiple computer systems distributed across a network and operating in a public or private cloud.

As shown in FIG. 1, the surgical planning and assistance device 12 includes a display 20. The display 20 may be embodied as any type of display capable of displaying digital images or other information, such as a liquid crystal display (LCD), a light emitting diode (LED), a plasma display, a cathode ray tube (CRT), or other type of display device. In some embodiments, the display 20 may be coupled to a touch screen to allow user interaction with the surgical planning and assistance device 12. Other user inputs, such as foot pedals, may also be used.

The surgical planning and assistance device 12 further includes one or more cameras 22. Each of the cameras 22 may be embodied as a digital camera or other digital imaging device coupled to the surgical planning and assistance device 12. Each camera 22 includes an electronic image sensor, such as an active-pixel sensor (APS), e.g., a complementary metal-oxide-semiconductor (CMOS) sensor, or a charge-coupled device (CCD). In the illustrative embodiment, multiple cameras 22 are arranged in an array and are thus capable of determining distance to objects imaged by the cameras 22.

The robotic assisted surgery device 14 may be embodied as any type of robot capable of performing the functions described herein. Illustratively, the robotic assisted surgery device 14 is embodied as a robotic arm that may be attached to a surgical table or otherwise positioned near a patient during the orthopaedic surgical procedure. The robotic assisted surgery device 14 includes a surgical tool 16, illustratively embodied as a surgical saw 16. In use, the illustrative robotic assisted surgery device 14 supports the surgical saw 16 and may constrain movement of the surgical saw 16 within a resection plane specified in a surgical plan (e.g., relative to a femur or tibia of the patient). The surgeon may activate the surgical saw 16 and perform the resection with the surgical saw 16 while the robotic assisted surgery device 14 constrains movement of the surgical saw 16 to the resection plane. The robotic assisted surgery device 14 may illustratively be embodied as a VELYS™ Robotic-Assisted Solution, commercially available from DePuy Synthes Products, Inc. of Warsaw, Indiana. Although illustrated with a surgical saw 16, it should be understood that, in other embodiments, the robotic assisted surgery device 14 may include, or be used with, one or more other surgical instruments, such as, for example, surgical burrs, chisels, impactors, reamers, and other powered surgical tools. Furthermore, while the present disclosure generally refers to surgical plans including a number of resection planes, it is also contemplated that the robotic assisted surgery device 14 could be used to perform one or more non-planar resections according to a surgical plan. For instance, in some embodiments, the robotic assisted surgery device 14 might guide a surgical burr to prepare a curved surface on a bone to receive a prosthesis.

The surgical planning and assistance device 12 and the robotic assisted surgery device 14 may be configured to transmit and receive data with each other and/or other devices of the system 10 over a network. The network may be embodied as any number of various wired and/or wireless networks. For example, the network may be embodied as, or otherwise include, a wired or wireless local area network (LAN), a wired or wireless wide area network (WAN), a cellular network, and/or a publicly-accessible, global network such as the Internet. As such, the network include any number of additional devices, such as additional computers, routers, stations, and switches, to facilitate communications among the devices of the system 10.

The system 10 further includes the tracking markers 18. As described further below, in use, the surgical planning and assistance device 12 may track the location of the tracking markers 18 in space using the array of cameras 22. For example, each tracking marker 18 may include a number of hydrophobic optical reflectors arranged in a predetermined pattern visible to the cameras 22. Illustratively, some of the tracking markers 18 are secured to the robotic assisted surgery device 14 and to the associated surgical tool 16, which allow the device 12 to track the location of the robotic assisted surgery device 14 and/or the surgical tool 16. As described further below, the system 10 may also track arrays secured to the patient's bones and a pointer that may be temporarily positioned by a surgeon relative to anatomical landmarks of the patient while the pointer is observed by the cameras 22. Although illustrated as including tracking markers 18 suitable for optical tracking with the cameras 22, it should be understood that, in other embodiments, the system 10 may use electromagnetic tracking or other position tracking technology for tracking the patient and instruments.

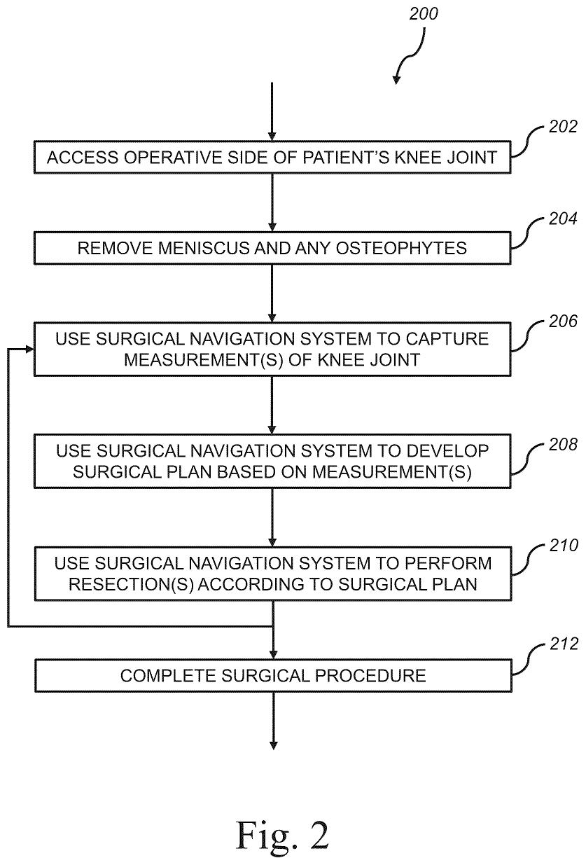

Referring now to FIG. 2, an orthopaedic surgeon may illustratively use the system 10 to perform a UKA surgical procedure according to the method 200. (Although illustrated herein in connection with a UKA surgical procedure, it should be understood that the system 10 may also be used with other orthopaedic surgical procedures, such as total knee arthroplasty, or TKA, surgical procedures.) The method 200 begins with block 202, in which the surgeon accesses the operative side of the patient's knee joint. Depending on the particular UKA surgical procedure, the operative side may be the medial side or the lateral side of the patient's knee joint. Unless otherwise noted, any depictions and/or descriptions herein referring to a medial side of a knee joint are equally applicable to a lateral side of a knee joint, and vice versa. Block 202 will typically involve the surgeon creating an incision anterior to the operative side of the patient's knee joint and moving soft tissues aside. After block 202, the method 200 proceeds to block 204, in which the surgeon removes the meniscus and any osteophytes that may be present in the joint space being resurfaced.

After completing block 204, the surgeon proceeds to block 206 of method 200. In block 206, the surgeon uses the system 10 to capture measurements of various features of the patient's knee joint. Block 206 may involve capturing one or more measurements on the operative side of a femur 24 of the patient relative to a femur coordinate space defined by the system 10, one or more measurements on the operative side of a tibia 26 of the patient relative to a tibia coordinate space defined by the system 10, and a spatial relationship between the femur coordinate space and the tibia coordinate space at each of a number of different poses of the knee joint, among other measurements. As discussed further below, block 206 may be performed prior to any resections being performed on the femur 24 or the tibia 26, but may also be performed after any number of resections have been performed in block 210, in order to determine the actual planes of those resections and/or to check whether the actual planes conform to the planned planes of the surgical plan.

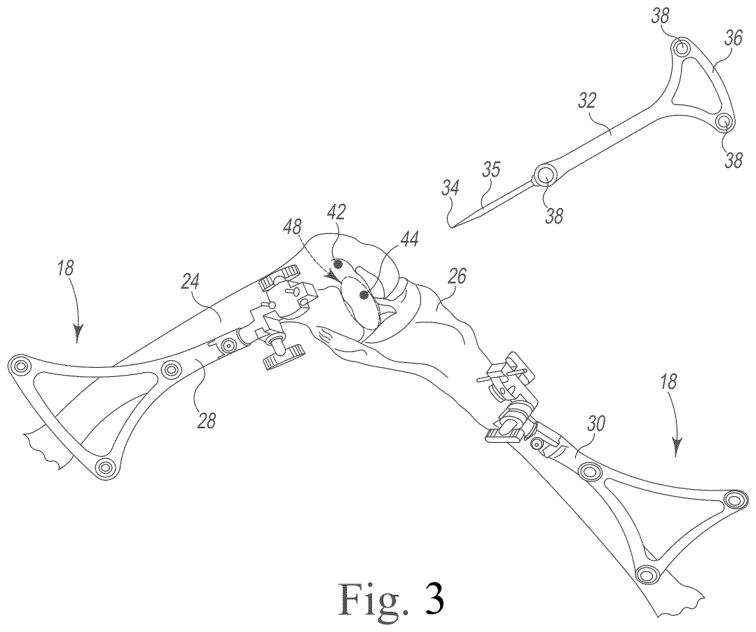

As shown in FIGS. 3-5, to capture these measurements, the surgeon attaches arrays 28, 30 (which include tracking markers 18) to the femur 24 and the tibia 26, respectively. The device 12 uses the cameras 22 to track the position of the bone arrays 28, 30 and thus register relative positions of the patient's femur 24 and tibia 26. The surgeon may manipulate the patient's femur 24 and tibia 26 in order to capture spatial relationships between the femur 24 and the tibia 26 over various ranges of motion. For example, the surgeon may rotate the femur 24 to acquire hip center. As another example, the surgeon may capture a spatial relationship between the femur 24 and the tibia 26 (as represented, for example, by a spatial relationship between the femur and tibia coordinate spaces) at each of a number of different poses throughout a range of motion from full extension to maximum flexion. For instance, the system 10 may capture a spatial relationship between the femur 24 and the tibia 26 when these bones are in full extension (i.e., 0 degrees flexion). The system 10 may also capture a spatial relationship between the femur 24 and the tibia 26 when these bones are flexed at 90 degrees to one another. In some embodiments, the system 10 may further capture a spatial relationship between the femur 24 and the tibia 26 at additional poses between 0 and 90 degrees flexion, which can be spaced at regular intervals. In the illustrative embodiment, the system 10 captures a spatial relationship between the femur 24 and the tibia 26 at every 5 degrees of flexion between 0 and 90 degrees. Using this spatial relationship data, combined with data relating to the shape of the femur 24 and the shape of the tibia 26 (such as the points and/or surfaces discussed below, or shape information derived therefrom), the system 10 may determine relationships between various features of the femur 24 and the tibia 26 (as well as prostheses positioned relative to the femur 24 and the tibia 26) at different poses throughout the range of motion.

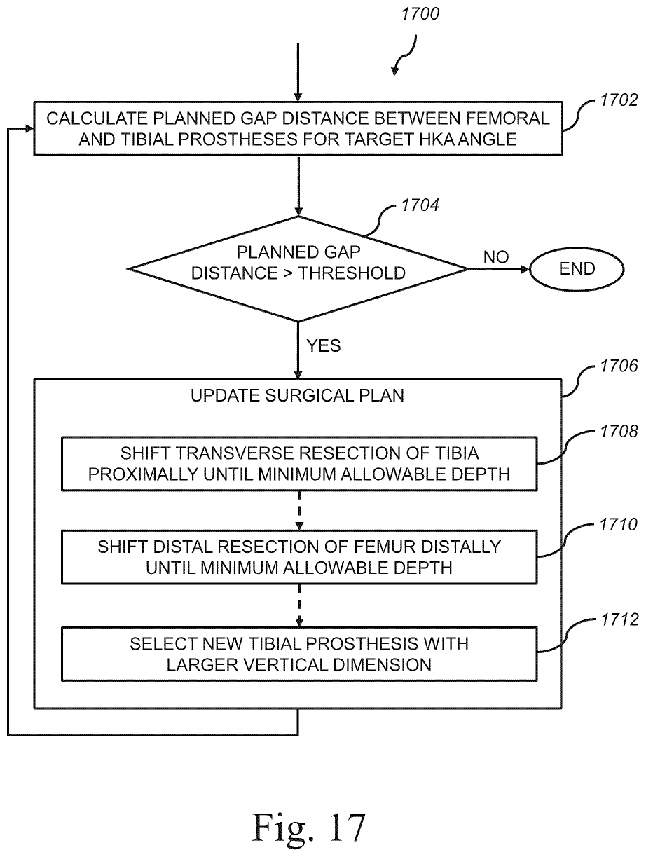

In the illustrative embodiment, block 206 also involves using the system 10 to capture a spatial relationship between the femur coordinate space and the tibia coordinate space while the surgeon positions the femur 24 and the tibia 26 in full extension with a target HKA angle. During this measurement, the surgeon places a varus or valgus stress on the knee joint to align the femur 24 and the tibia 26 in the surgeon's desired HKA angle for the patient. As discussed below (specifically in relation to FIG. 17), the illustrative embodiment of the method 200 aims to achieve this target HKA angle when developing the surgical plan in block 208.

The surgeon also uses a pointer 32 to capture various points on the patient's bony anatomy. The pointer 32 includes tracking markers 18 and can function as a registration tool for registering anatomical landmarks of the patient's bony anatomy during the orthopaedic surgical procedure. The illustrative pointer 32 includes an elongated body 35 that extends from a tip 34 to a triangular frame 36. The frame 36 supports hydrophobic optical reflectors 38, which may be tracked by the device 12 using the cameras 22 as described above. In use, the surgeon positions the tip 34 (or elongated body 35) of the pointer 32 in a desired location. The device 12, using the camera 22, tracks the location of the reflectors 38 and, based on those tracked locations, determines the corresponding position of the tip 34 (or elongated body 35).

By way of example, the surgeon may create a tibial checkpoint and then acquire the position of the tibial checkpoint in the tibia coordinate space with the pointer 32. The surgeon may also create a femur checkpoint and acquire the position of the femur checkpoint in the femur coordinate space with the pointer 32. The surgeon may further acquire the positions of the medial malleolus and the lateral malleolus with the pointer 32. As shown in FIGS. 3 and 4, the surgeon may use the pointer 32 to capture positions in the femur coordinate space of a femur center 40, an anterior sizing point 42 on the operative side of the femur 24, a most distal point 44 on the operative side of the femur 24, and/or a most posterior point 46 on the operative side of the femur 24. As best seen in FIG. 3, the surgeon may also use the pointer 32 to acquire a femoral condyle surface 48 relative to the femur coordinate space. To capture the femoral condyle surface 48, the surgeon may move the tip 34 of the pointer 32 across the surface 48, and the system 10 may capture multiple positions on the surface 48. Those captured positions may be represented as a point cloud or other collection of location data associated with the femoral condyle surface 48. In some embodiments, the system 10 may automatically choose the most distal point 44 and the most posterior point 46 (on the operative side of the femur 24) from among the points defining the surface 48.

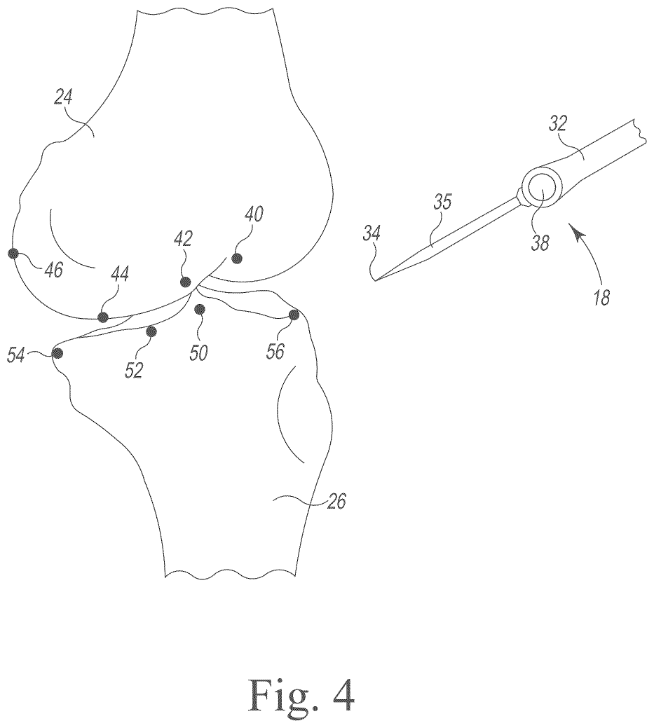

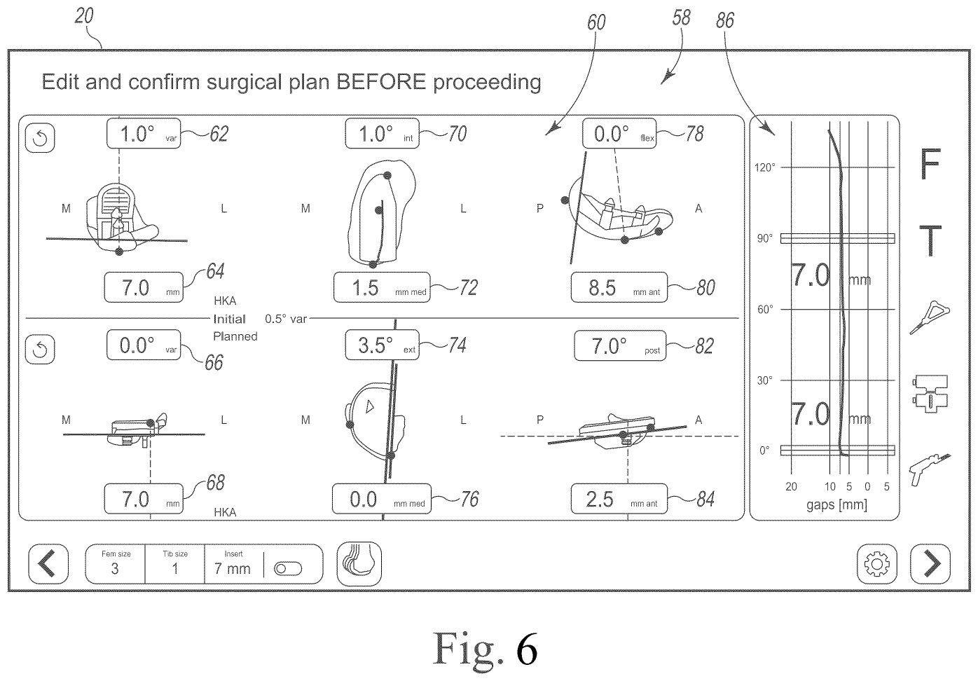

The surgeon may also capture various points on the patient's tibia 26 in block 206. As shown in FIGS. 4 and 5, the surgeon may use the pointer 32 to acquire positions in the tibia coordinate space of a tibia center 50, a most anterior point 52 on the operative side of the tibia 26 (at the level of either the anticipated transverse resection or the actual transverse resection), a most collateral point 54, 56 on the operative side of the tibia 26 (either a most medial point 54 or a most lateral point 56, depending on the operative side of the tibia), a most posterior point 92 on the operative side of the tibia 26, and/or a resection reference point 94 on the operative side of the tibia 26. Prior to the transverse resection of the tibia 26 being performed, the most collateral point 54, 56 may be captured by touching the tip 34 of the pointer 32 to a point in an anticipated plane 106 of the transverse resection of the tibia 26 (see FIG. 7). After the transverse resection of the tibia 26 has been performed, the most collateral point 54, 56 may be acquired (or reacquired) by touching the tip 34 of the pointer 32 to a point on a transverse resected surface 118 of the tibia 26 (i.e., in an actual plane of the transverse resection).

Block 206 may also involve the surgeon using the pointer 32 to measure various lines and/or surfaces of in the tibia coordinate space. For instance, the surgeon may use the pointer 32 to capture a tibial plateau surface 96. Similar to the femoral condyle surface 48 described above, the surgeon may move the tip 34 of the pointer 32 across the surface 96, and the system 10 may capture multiple positions on the surface 96. Those captured positions may be represented as a point cloud or other collection of location data associated with the tibial plateau surface 96. In some embodiments, the system 10 may automatically choose the resection reference point 94 (on the operative side of the tibia 26) from among the points defining the surface 48 (e.g., a most distal point from among those points).

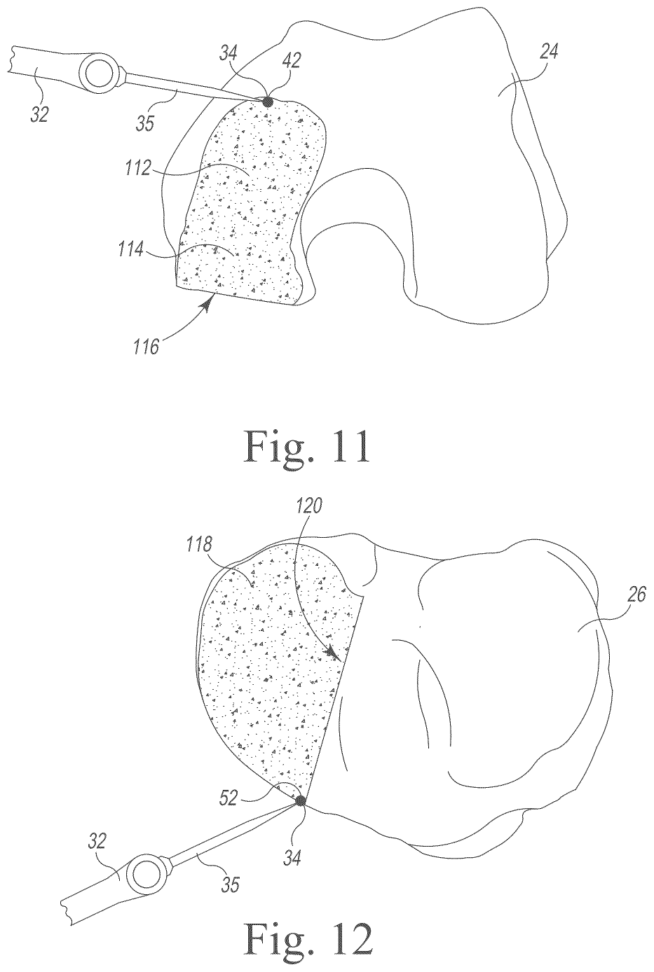

As illustrated in FIG. 5, the surgeon may also use the pointer 32 to capture a tibial compartment border 98 on the operative side of the tibia 26. Prior to the transverse and sagittal resections of the tibia 26 being performed, the surgeon may align the elongated body 35 of the pointer 32 just above the tibial plateau surface 96 at the anticipated location of the sagittal resection of the tibia 26. The system 10 then captures the position and orientation of the elongated body 35 of the pointer 32 relative to the tibia coordinate space to define the tibial compartment border 98. The surgeon may additionally or alternatively use the pointer 32 to capture an actual plane of a sagittal resected surface 120 of the tibia 26 after the transverse and sagittal resections of the tibia 26 have been performed in block 210. To do so, the surgeon can move the tip 34 of the pointer 32 along the sagittal resected surface 120 (see FIG. 12), and the system 10 may capture multiple positions on the surface 120. Those captured positions, which may be represented as a point cloud or other collection of location data associated with the sagittal resected surface 120, may then be recorded as the actual (post-resection) tibial compartment border 98. Additionally or alternatively, the actual plane of the sagittal resected surface 120 of the tibia 26 might be registered by placing the elongated body 35 of the pointer 32 on the sagittal resected surface 120 while the system 10 captures the position and orientation of the pointer 32 relative to the tibia coordinate space.

In some embodiments, capturing the anterior sizing point 42 on the operative side of the femur 24 (discussed above) involves placing the femur 24 and the tibia 26 into full extension, and then touching the tip 34 of the pointer 32 to a point on the operative side of the femur 24 that is directly proximal of the most anterior point 52 of the tibia 26. If the transverse resection of the tibia 26 has not yet been performed, the most anterior point 52 is located in the plane of the anticipated transverse resection. However, if the transverse resection of the tibia 26 has already been performed, the most anterior point 52 is located in the plane of the actual transverse resection (i.e., the most anterior point 52 of the transverse resected surface 118, see FIG. 12).

It will be appreciated that, in other embodiments, the surgeon may capture a different number and/or arrangement of bony anatomy landmarks or other features. Furthermore, although illustrated as capturing measurements of the knee joint using the pointer 32, it should be understood that, in some embodiments, the system 10 may additionally or alternatively capture measurements of the knee joint using a laser scanner, a white light scanner, a structured light scanner, or other surface scanner. As another example, the system 10 may additionally or alternatively capture measurements of the knee joint using an ultrasound imaging scanner or other volumetric scanner.

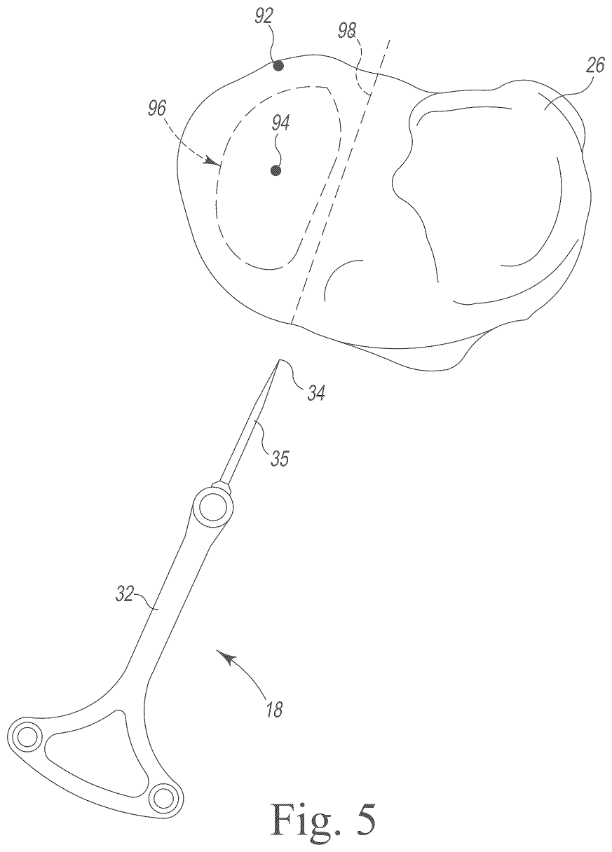

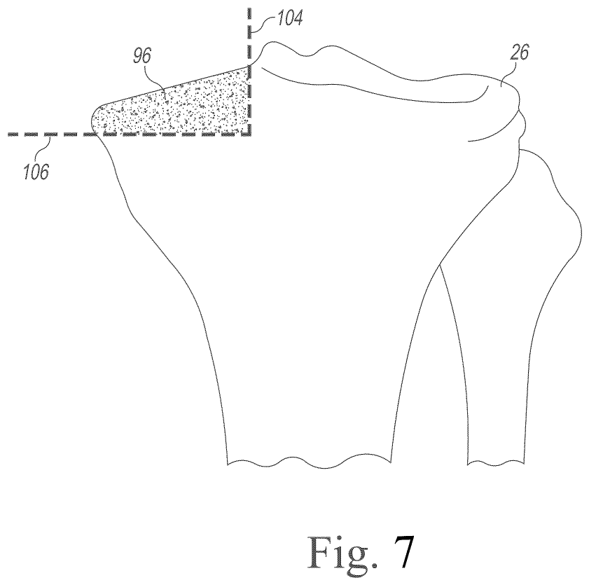

Referring back to FIG. 2, after block 206, the surgeon proceeds to block 208 of method 200. In block 208, the surgeon uses the system 10 to develop a surgical plan for the UKA surgical procedure based on the measurements captured in block 206. As used herein, “develop(ing) a surgical plan” (and similar phrases) may refer to creating a new surgical plan and/or modifying an already existing surgical plan. The surgical plan includes a number of parameters, such as planned (or actual) planes for resections of the femur 24 and of the tibia 26, as well as selected sizes and planned positions for the femoral and tibial prostheses to be implanted on the resected surfaces created by the resections. The position of the tibial prosthesis (in the tibia coordinate space) and the position of the femoral prosthesis (in the femur coordinate space) can each be planned in up to six degrees of freedom, including medial-lateral translation, proximal-distal translation, anterior-posterior translation, internal-external rotation, varus-valgus rotation, and flexion-extension rotation (see FIG. 6). The surgical plan also specifies a selected size (from among a plurality of possible sizes) for the femoral prosthesis and for the tibial prosthesis, as well as various dimensions of the selected prosthesis. With regard to the tibial prosthesis, the surgical plan may include selected sizes for both a tibial tray and a bearing insert that combine to make the tibial prosthesis, and relevant dimensions. The surgical plan includes a sagittal resection plane 104 and a transverse resection plane 106 defined relative to the tibia coordinate space (see FIG. 7). The surgical plan also includes a posterior resection plane 88, a distal resection plane 108, and a posterior chamfer resection plane 110 defined relative to the femur coordinate space (see FIGS. 8-10; the positions of these planes are determined by the selected size and planned position of the femoral prosthesis relative to the femur coordinate space).

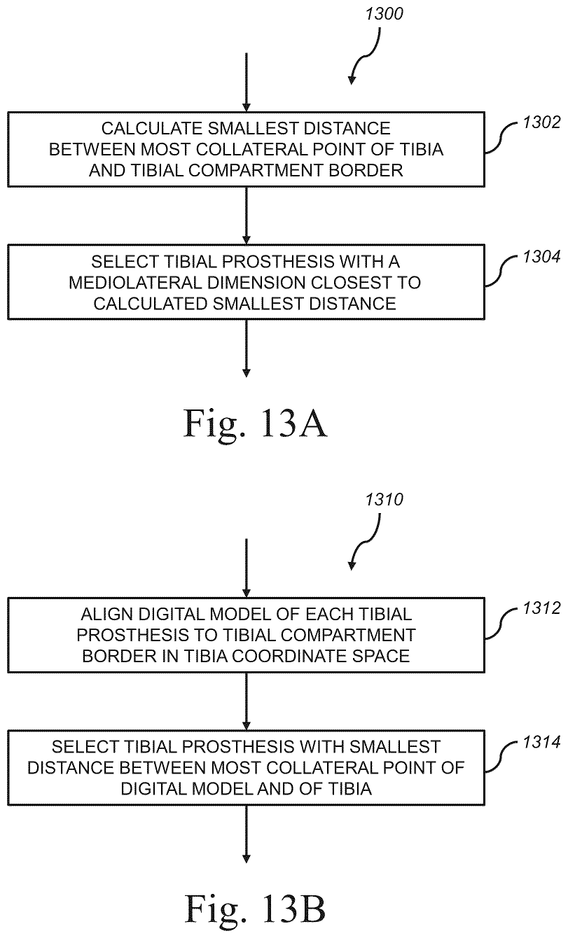

In block 208, the system 10 initially determines the surgical parameters of the surgical plan based on the measurements captured in block 206, default values, constraints, and automatic optimization of certain parameters. Various optimizations of the surgical plan that can be performed by the system 10, automatically and/or when requested by the user (e.g., after modifying or performing some aspect of the surgical plan), are discussed in more detail below with reference to FIGS. 13A-17. The surgical plan initially determined by the system 10 in block 208 is presented to the surgeon for review and refinement. FIG. 6 shows one illustrative user interface 58 that may be displayed on the display 20 of the device 12 and may be used by the surgeon to develop the surgical plan in block 208. The user interface 58 includes an interactive representation 60 of the surgical plan. As shown, the interactive representation 60 includes graphical representations of the patient's anatomy and associated femoral and tibial prostheses. The interactive representation 60 also includes textual and/or numerical representations of various surgical parameters. Those surgical parameters may be interactively viewed and/or edited by the surgeon or other user in block 208, before continuing with the UKA surgical procedure of method 200.

Illustratively, the interactive representation 60 includes user interface elements for viewing and/or editing various surgical parameters defining the position of the femoral and tibial prosthesis relative to their respective coordinate spaces: a femoral prosthesis varus-valgus angle 62, a posterior femoral resection height 64, a tibial prosthesis varus-valgus angle 66, a tibial resection height 68, a femoral prosthesis internal-external rotation angle 70, a femoral prosthesis medial-lateral shift 72, a tibial prosthesis internal-external rotation angle 74, a tibial prosthesis medial-lateral shift 76, a femoral prosthesis flexion-extension angle 78, a femoral prosthesis anterior-posterior shift 80, a tibial slope angle 82, and a tibial prosthesis anterior-posterior shift 84. The interactive representation 60 may further include additional user interface elements for other information or surgical parameters, including HKA angle, mechanical or kinematic alignment, sizes of the femoral prosthesis and tibial prosthesis (including tibial tray and bearing insert), and other information.

As shown, the user interface 58 further includes a joint balance graph 86, which is an interactive representation of a joint gap on the operative side of the patient's knee joint through a range of motion (e.g., 0-120 degrees of flexion). Illustratively, the joint balance graph 86 in FIG. 6 includes a graphical representation of calculated values of a gap between the femoral prosthesis and the planned transverse resection of the tibia) based on measurements captured in block 206 and current values of various surgical parameters of the surgical plan, including the selected size of the femoral prosthesis. The joint balance graph 86 may be updated based on new measurements captured in block 206 and/or changes to the surgical plan. For example, adjusting the position of the planned transverse resection plane 106 may change the values shown in the joint balance graph 86. Similarly, measuring a position of the transverse resected surface 118 (after the transverse resection has been performed) and updating the surgical plan to match the actual plane of the tibia transverse resection may also change the values shown in the joint balance graph 86. The surgeon confirms the surgical plan when the surgical parameters of the surgical plan and the joint balance graph 86 are satisfactory.

Referring back to FIG. 2, after developing a surgical plan in block 208, the surgeon proceeds to block 210 of method 200. In block 210, the surgeon uses the system 10 to perform resections of the femur 24 and/or the tibia 26 of the patient's knee joint according to the surgical plan. As further discussed below, in some embodiments, block 210 may involve performing only a subset of the planned resections of the surgical plan (for instance, the planned resections of the tibia 26) and then looping back to block 206 to capture additional measurements.

In the illustrative embodiment, block 210 involves the surgeon performing two resections of the tibia 26 according to the surgical plan, as suggested in FIG. 7. Just before resecting the tibia 26, the surgeon may place the tip 34 of the pointer 32 on the previously marked checkpoint on the tibia 26 to verify that the tibia array 30 has not moved. The device 12 may display a representation of the relative locations of the tip 34 and the tibia checkpoint, and may indicate whether the tip 34 is positioned on the predetermined position of the tibia checkpoint. For example, the display 22 may include a graphical representation of the relative locations of the tip 34 and the tibia 26 and a numerical representation of the distance between the current location of the tip 34 and the tibia checkpoint.

Once the surgeon verifies the location of the tibia checkpoint, the surgeon uses the robotic assisted surgery device 14 to perform the transverse resection of the tibia 26. The robotic assisted surgery device 14 positions the surgical saw 16 in the transverse resection plane 106. The transverse resection plane 106 is defined in the surgical plan relative to the tibia coordinate space, as discussed above. The transverse resection plane 106 may also be measured relative to the tibial resection reference point 94 and/or the tibial plateau surface 96. For example, the transverse resection plane 106 may be positioned at a certain distance in millimeters from the tibial resection reference point 94, i.e., the tibial resection height 68. The transverse resection plane 106 may be positioned at an angle defined by the tibial slope angle 74. The robotic assisted surgery device 14 supports the surgical saw 16 and may constrain movement of the surgical saw 16 within the transverse resection plane 106 while the surgeon uses the surgical saw 16 to perform the transverse resection of the tibia 26.

Additionally, either before or after the transverse resection of the tibia 26, the surgeon uses a reciprocating saw to perform the sagittal resection of the tibia 26. In the illustrative embodiment, the surgeon couples a reciprocating saw handpiece (not shown) to the robotic assisted surgery device 14 and then manually positions the reciprocating saw blade in the sagittal resection plane 104. The sagittal resection plane 104 is defined in the surgical plan relative to the tibia coordinate space, as discussed above. The sagittal resection plane 104 may also be positioned at a certain distance from a landmark of the tibia 26, and may be angled at a certain rotation angle, such as the tibial prosthesis rotation angle 82. After manually positioning the reciprocating saw blade, the robotic assisted surgery device 14 moves down and allows the surgeon to perform the sagittal resection of the tibia 26 if the reciprocating saw blade is within an acceptable range of the sagittal resection plane 104.

In some embodiments, after performing the transverse and sagittal resections of the tibia 26, the method 200 may loop back to block 206 in which the surgeon performs one or more measurements on the surgically prepared tibia 26. For instance, as illustrated in FIG. 12, the surgeon may place the tip 34 of the pointer 32 on one or more points on the transverse resected surface 118 and/or the sagittal resected surface 120 of the tibia 26, while the system 10 captures the position of the pointer 32. The surgeon may also move the tip 34 of the pointer 32 along portions of the transverse resected surface 118 and/or the sagittal resected surface 120 to measure these resected surfaces in the tibia coordinate space.

In these embodiments, the method 200 will then proceed again to block 208, in which the system 10 compares the measurements of the transverse resected surface 118 to the planned transverse resection plane 106 of the surgical plan and/or compares the sagittal resected surface 120 to the planned sagittal resection plane 104 of the surgical plan, in order to confirm compliance with the surgical plan. To the extent any measurements of the resected surfaces 118, 120 differ materially from the planned resection planes 104, 106, the surgical plan can be updated in block 208. For example, the resection planes 104, 106 of the surgical plan can be updated to conform to the measurements of the resected surfaces 118, 120, and the remaining parameters of the surgical plan can be re-optimized (while the resection planes 104, 106 are constrained to their actual positions in the tibia coordinate space). The surgeon may also use the user interface 58 and the joint balance graph 86 to assess and/or modify the surgical plan, as described above. The surgeon can confirm the surgical plan when the surgical parameters of the surgical plan and the joint balance graph 86 are satisfactory.

Referring now to FIGS. 8-10, in the illustrative embodiment, after performing the transverse and sagittal resections of the tibia 26 (and, optionally, after looping back to blocks 206 and 208, as just discussed), the surgeon next uses the robotic assisted surgery device 14 to perform three resections of the femur 24 according to the surgical plan: the posterior resection, the distal resection, and the posterior chamfer resection of the femur 24. Just before resecting the femur 24, the surgeon may place the tip 34 of the pointer 32 on the previously marked checkpoint on the femur 24 to verify that the femur array 28 has not moved. The device 12 may display a representation of the relative locations of the tip 34 and the femur checkpoint, and may indicate whether the tip 34 is positioned on the predetermined position of the femur checkpoint. For example, the display 22 may include a graphical representation of the relative locations of the tip 34 and the femur 24 and a numerical representation of the distance between the current location of the tip 34 and the femur checkpoint.

Once the surgeon verifies the location of the femur checkpoint, the robotic assisted surgery device 14 positions the surgical saw 16 in one of the femoral resection planes 88, 108, 110 of the surgical plan. In the embodiment illustrated in FIGS. 8-10, the robotic assisted surgery device 14 first positions the surgical saw 16 in the posterior resection plane 88 shown in FIG. 8. The posterior resection plane 88 is defined in the surgical plan relative to the femur coordinate space, as discussed above. The posterior resection plane 88 may also be measured relative to a posterior condyle surface 90. For example, the posterior femoral resection plane 88 may be positioned at a certain distance in millimeters from the most posterior point of the posterior condyle 90 extracted from the posterior condyle acquisition, i.e., the posterior femoral resection height 64. The robotic assisted surgery device 14 supports the surgical saw 16 and may constrain movement of the surgical saw 16 within the posterior resection plane 88 while the surgeon uses the surgical saw 16 to perform the posterior resection of the femur 24.

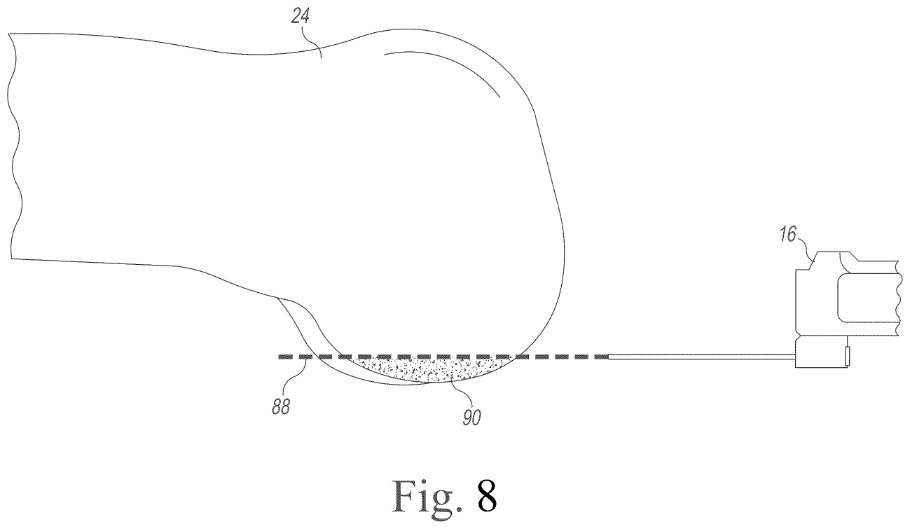

In the embodiment illustrated in FIGS. 8-10, the robotic assisted surgery device 14 next positions the surgical saw 16 in the distal resection plane 108 shown in FIG. 9. The distal resection plane 108 is defined in the surgical plan relative to the femur coordinate space, as discussed above. The height and/or angle of the distal resection plane 108 may be determined based on the femoral prosthesis flexion-extension angle 70 and/or other surgical parameters of the surgical plan. The robotic assisted surgery device 14 supports the surgical saw 16 and may constrain movement of the surgical saw 16 within the distal resection plane 108 while the surgeon uses the surgical saw 16 to perform the distal resection of the femur 24.

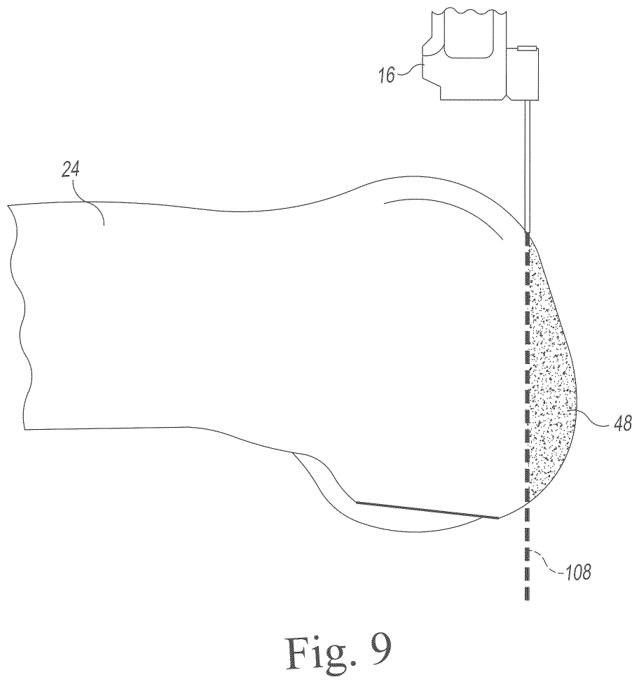

In the embodiment illustrated in FIGS. 8-10, the robotic assisted surgery device 14 next positions the surgical saw 16 in the posterior chamfer resection plane 110 shown in FIG. 10. The posterior chamfer resection plane 110 is defined in the surgical plan relative to the femur coordinate space, as discussed above. The robotic assisted surgery device 14 supports the surgical saw 16 and may constrain movement of the surgical saw 16 within the posterior chamfer resection plane 110 while the surgeon uses the surgical saw 16 to perform the posterior chamfer resection.

It is contemplated that the resections of the femur 24 and the tibia 26 described above may be performed in different orders in other embodiments. For instance, in one illustrative embodiment, the surgeon may begin with the transverse resection of the tibia 26 (FIG. 7), followed by the sagittal resection of the tibia 26 (FIG. 7), then the distal resection of the femur 24 (FIG. 9), then the posterior chamfer resection of the femur 24 (FIG. 10), and finally the posterior resection of the femur 24 (FIG. 8). Other embodiments may utilize any of the femoral resections as the first resection, as described in U.S. patent application Ser. No. 18/232,388, filed Aug. 10, 2023 (the entire disclosure of which is incorporated herein by reference). In such embodiments, after capturing an initial set of measurements (block 206) and developing a surgical plan based on that initial set of measurements (block 208), the surgeon may proceed to use the robotic assisted surgery device 14 to perform a first femoral resection in substantially the same manner as described above with reference to any of FIGS. 8-10 (block 210). The method 200 may then return to block 206, for the surgeon to capture one or more additional measurements on portions of the patient's knee joint that were previously inaccessible due to tightness of the knee (i.e., the tibia 26 and the femur 24 being in close contact throughout their entire range of motion) but that are currently accessible after performing the first femoral resection. After completing these one or more additional measurements (block 206), the surgeon may confirm/update the surgical plan based on the additional measurement(s) (block 208) and then perform additional resections according to the confirmed/updated surgical plan (block 210).

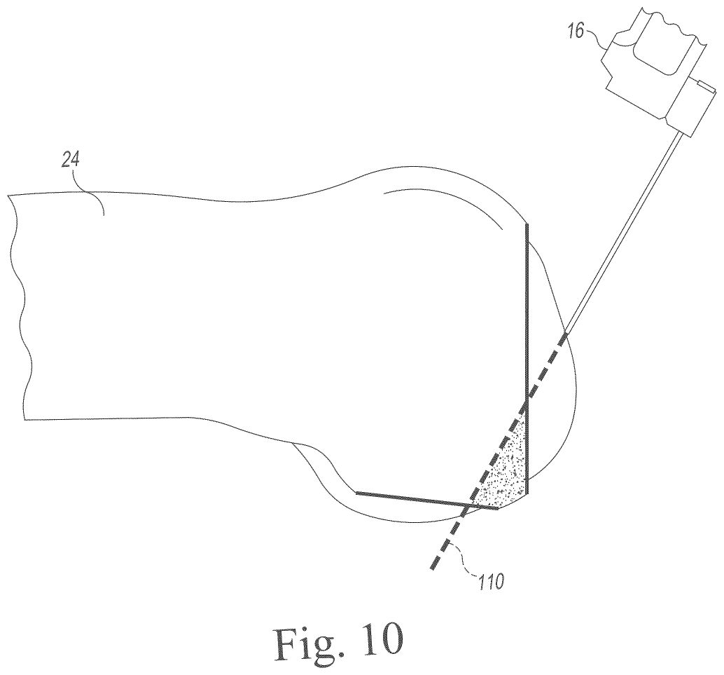

Referring back to FIG. 2, after performing one or more resections in block 210, the surgeon proceeds to block 212 of method 200 and completes the UKA surgical procedure. In the illustrative embodiment, block 212 involves the surgeon using the system 10 to make one or more reference markings on the femur 24. For instance, as shown in FIG. 11, the surgeon can place the tip 34 of the pointer 32 on a distal resected surface 112 of the femur 24, while the system 10 captures the position of the pointer 32. The device 12 may display a representation of the relative locations of the tip 34 and the femur 24 to indicate whether the tip 34 is positioned on a previously measured/planned position for a particular point, line, or plane (e.g., the femoral anterior sizing point 42, a mediolateral centerline of distal resected surface 112, the plane 108, etc.). For example, the display 22 may include a graphical representation of the relative locations of the tip 34 and the femur 24 and a numerical representation of the distance between the current location of the tip 34 and the point or line to be marked by the surgeon. By way of example, when the tip 34 is positioned at the femoral anterior sizing point 42, as shown in FIG. 11, the surgeon may mark this point on the femur 24 (e.g., using a writing instrument). In another illustrative embodiment, the surgeon can use the system 10 to mark a plurality of points along the mediolateral centerline of distal resected surface 112 and then draw a line connecting those points (e.g., using a writing instrument). During such marking, the device 12 may display a graphical representation of the mediolateral centerline superimposed on the femur 24 for confirmation by the surgeon.

The surgeon may then continue performing the orthopaedic surgical procedure using one or more manual instruments. For example, the surgeon may align a femoral finishing block, cutting guide, or jig with the marking(s) made on the distal resected surface 112 and then use that instrument to drill, ream, or otherwise peg holes in the femur 24. Additionally or alternatively, the surgeon may use the system 10 to verify the accuracy of any aspect(s) of the distal resected surface 112, the posterior chamfer resected surface 114 of the femur 24, and/or the posterior resected surface 116 of the femur 24 (using a similar approach to that described above).

In the illustrative embodiment, block 212 involves the surgeon using the system 10 to make one or more reference markings on the tibia 26. For instance, as shown in FIG. 12, the surgeon can place the tip 34 of the pointer 32 on the transverse resected surface 118 of the tibia 26, while the system 10 captures the position of the pointer 32. The device 12 may display a representation of the relative locations of the tip 34 and the tibia 26 to indicate whether the tip 34 is positioned on a previously measured/planned position for a particular point, line, or plane (e.g., the tibial most anterior point 52, the plane 106, etc.). For example, the display 22 may include a graphical representation of the relative locations of the tip 34 and the tibia 26 and a numerical representation of the distance between the current location of the tip 34 and the point or line to be marked by the surgeon. By way of example, when the tip 34 is positioned at the tibial most anterior point 52, as shown in FIG. 12, the surgeon may mark this point on the tibia 26 (e.g., using a writing instrument).

The surgeon may then continue performing the orthopaedic surgical procedure using one or more manual instruments. For example, the surgeon may align a tibial template, cutting guide, or jig with the marking(s) made on the transverse resected surface 118 and then use that instrument to drill, ream, broach, cut, or otherwise prepare a peg hole and a keel slot in the tibia 26. Additionally or alternatively, the surgeon may use the system 10 to verify accuracy of any aspect(s) of the transverse resected surface 118 and/or the sagittal resected surface 120 of the tibia 26 (using a similar approach to that described above).

Block 212 may also involve the surgeon attaching one or more trial prostheses to the femur 24 and the tibia 26. The surgeon may use the system 10 to assess leg alignment and balance as described above in connection with FIG. 6. In block 212, the surgeon implants a final femoral prosthesis and a final tibial prosthesis (which may include a tibial tray coupled with a bearing insert, as discussed above). The surgeon may use the system 10 to assess final leg alignment and joint balance as described above in connection with FIG. 6. Block 212 and method 200 conclude with the surgeon removing all surgical instruments and closing the knee joint.

In the illustrative embodiment, the system 10 is configured to optimize various surgical parameters of the surgical plan to assist the surgeon in developing the surgical plan in block 208 of the method 200 of FIG. 2. The optimizations, several of which are illustrated in FIGS. 13A-17 and described in detail below, may be run automatically by the system 10 when initializing a new surgical plan in block 208 and/or when requested by the user (e.g., after modifying some aspect of the surgical plan, or after performing some aspect of the surgical plan and capturing measurements of the resulting resection(s)). In the latter case, the system 10 treats any surgical parameters modified by the user and/or any surgical parameters measured from already performed resections as fixed and attempts to optimize the remaining surgical parameters of the surgical plan.

In the illustrative embodiment, the system 10 is operable to automatically select the surgical plan's size of the tibial tray from among the possible sizes for the tibial tray based on the most collateral point 54, 56 and the tibial compartment border 98 captured during block 206 of method 200. As noted above, the most collateral point 54, 56 and the tibial compartment border 98 may be registered based on anticipated resection planes 104, 106 (before resection of the tibia 26) or actual resected surfaces 118, 120 (after resection of the tibia 26). FIG. 13A illustrates one method 1300 for selecting the surgical plan's size of the tibial tray that may be performed by the system 10, while FIG. 13B illustrates an alternative method 1310 for selecting the surgical plan's size of the tibial tray that may be performed by the system 10.

The methods 1300, 1310 can each be performed before or after the tibia 26 has been resected. When performed before the tibia 26 has been resected, the methods 1300, 1310 utilize a planned position for the tibial tray, which may be based on default positioning parameters, surgeon-modified positioning parameters, system-optimized positioning parameters, or a combination thereof. In the illustrative embodiment, the default positioning parameters for the tibial tray include a resection depth of 7 mm for the transverse resection plane 106, a 7 degree posterior slope, no varus-valgus rotation, an anterior-posterior translation aligned to the tibial most anterior point 52, and both a medial-lateral translation and an internal-external rotation that are aligned to the tibial compartment border 98. Any or all of these positioning parameters for the tibial prosthesis may be modified manually by the surgeon using the user interface 58 and/or automatically by the system 10 as part of one of the planning algorithms described herein (e.g., as part of method 1700 of FIG. 17, described in detail below).

The method 1300 of FIG. 13A begins with block 1302, in which the system 10 calculates a smallest distance in the tibia coordinate space between the most collateral point 54, 56 (depending on the operative side) and the tibial compartment border 98. After block 1302, the method 1300 proceeds to block 1304, in which the system 10 selects, from among the possible sizes for the tibial tray, the possible size with the mediolateral dimension closest to the smallest distance previously calculated. At block 1304, the system 10 may also select, from among the possible sizes for the tibial tray, the possible size with the largest mediolateral dimension that does not exceed the smallest distance by more than one-half of a resolution of the system 10. In one embodiment, the resolution of the system 10 is 0.5 mm, so the system 10 selects the tibial tray with the largest mediolateral dimension closest to the calculated distance between the most collateral point 54, 56 and the tibial compartment border 98. In the illustrative embodiment, the resolution of the system 10 is 0.5 mm, so the system 10 selects the tibial tray with largest mediolateral dimension that does not exceed the calculated distance between the most collateral point 54, 56 and the tibial compartment border 98 by, for example, more than 0.25 mm in block 1304.

The method 1310 of FIG. 13B begins with block 1312, in which the system 10 aligns, in the tibia coordinate space, a digital model of each of the possible sizes for the tibial tray to the tibial compartment border 98. After block 1312, the method 1310 proceeds to block 1314, in which the system 10 selects the size of the tibial tray for the surgical plan by identifying the digital model (aligned to the tibial compartment border 98 in the tibia coordinate space) with smallest absolute distance between the most collateral point 54, 56 of the tibia 26 and a most collateral point of the digital model.

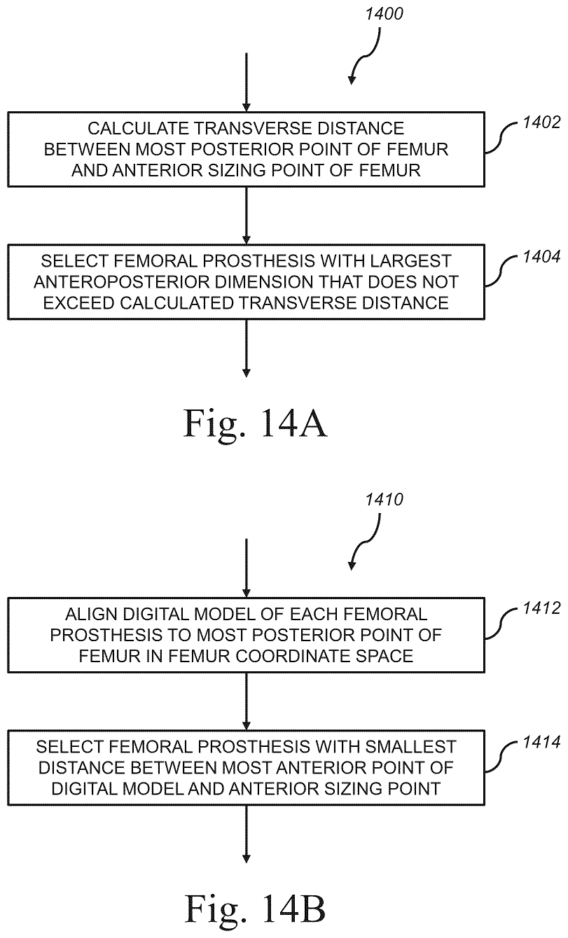

In the illustrative embodiment, the system 10 is also operable to automatically select the surgical plan's size of the femoral prosthesis from among the possible sizes for the femoral prosthesis based on the most posterior point 46 and the anterior sizing point 42 captured during block 206 of method 200. As noted above, the anterior sizing point 42 may be determined relative to the most anterior point 52 of the tibia 26, which may be registered based on an anticipated resection planes 106 (before resection of the tibia 26) or actual resected surfaces 118 (after resection of the tibia 26). FIG. 14A illustrates one method 1400 for selecting the surgical plan's size of the femoral prosthesis that may be performed by the system 10, while FIG. 14B illustrates an alternative method 1410 for selecting the surgical plan's size of the femoral prosthesis that may be performed by the system 10.

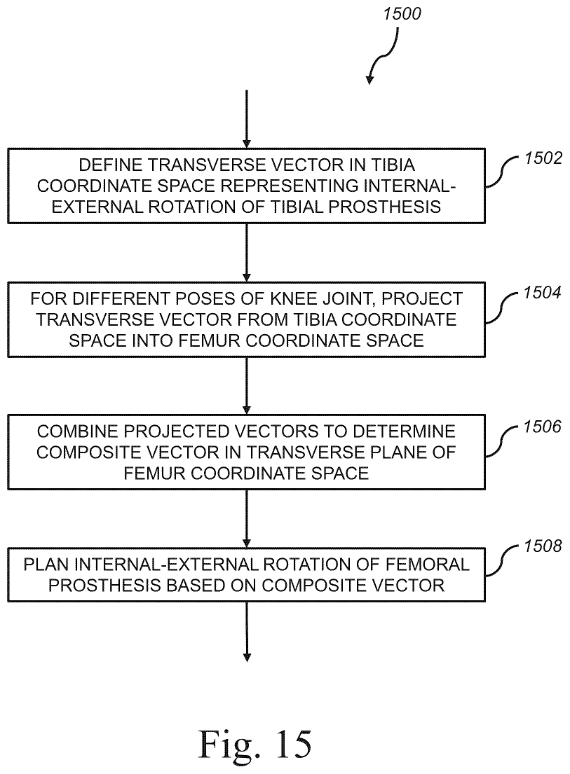

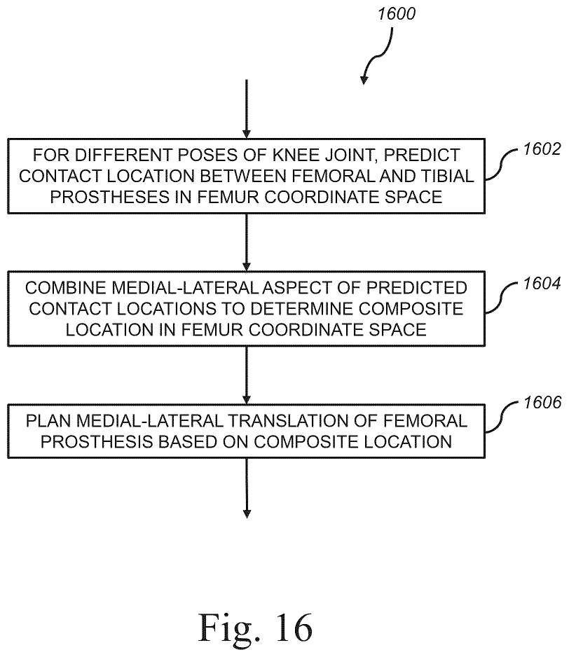

The methods 1400, 1410 each utilize a planned position for the femoral prosthesis, which may be based on default positioning parameters, surgeon-modified positioning parameters, system-optimized positioning parameters, or a combination thereof. In the illustrative embodiment, the default positioning parameters for the femoral prosthesis include a resection depth of 6.5 mm for the distal resection plane 108, a resection depth of 7.5 mm for the posterior resection plane 88, and both flexion-extension and varus-valgus rotations that set the femoral distal resection plane 108 parallel to the tibia transverse resection plane 106 when the femur 24 and tibia 26 are positioned in full extension with the target HKA angle captured in block 206. Additionally, in the illustrative embodiment, the internal-external rotation of the femoral prosthesis is chosen using the method 1500 of FIG. 15 (described in detail below), while the medial-lateral translation of the femoral prosthesis is chosen using the method 1600 of FIG. 16 (described in detail below). Alternatively, the methods 1400, 1410 may be performed using default positioning parameters for the internal-external rotation and medial-lateral translation of the femoral prosthesis. Furthermore, any or all of these positioning parameters for the femoral prosthesis may be modified manually by the surgeon using the user interface 58 and/or automatically by the system 10 as part of one of the planning algorithms described herein (e.g., as part of method 1500 of FIG. 15, method 1600 of FIG. 16, and/or method 1700 of FIG. 17, each described in detail below).

The method 1400 of FIG. 14A begins with block 1402, in which the system 10 calculates a transverse distance in the femur coordinate space between the most posterior point 46 and the anterior sizing point 42. In the illustrative embodiment, this transverse distance is parallel to a planned orientation for the anterior-posterior axis of the femoral prosthesis (either from the method 1500 of FIG. 15 or a default value). After block 1402, the method 1400 proceeds to block 1404, in which the system 10 selects, from among the possible sizes for the femoral prosthesis, the possible size with the largest anteroposterior dimension closest to the transvers distance or that does not exceed the transverse distance by, for example, more than one-half of a resolution of the system 10. In the illustrative embodiment, the resolution of the system 10 is 0.5 mm, so the system 10 selects the femoral prosthesis with the largest anteroposterior dimension closest to the calculated transverse distance or that does not exceed the calculated transverse distance between the most posterior point 46 and the anterior sizing point 42 by, for example, more than 0.25 mm in block 1404.

The method 1410 of FIG. 14B begins with block 1412, in which the system 10 aligns, in the femur coordinate space, a digital model of each of the possible sizes for the femoral prosthesis to the most posterior point 46. In the illustrative embodiment, each digital model is aligned using the system-optimized positioning parameters output by the methods 1500, 1600 (described in detail below) together with the default positioning parameters described above. After block 1412, the method 1410 proceeds to block 1414, in which the system 10 selects the size of the femoral prosthesis for the surgical plan by identifying the digital model (aligned to the most posterior point 46 in the femur coordinate space) with smallest distance between the anterior sizing point 42 and a most anterior point of the digital model. Both the method 1400 and the alternative method 1410 can be performed before or after the tibia 26 has been resected (which may change the size of the tibial prosthesis and therefore affect the size of the femoral prosthesis).

In the illustrative embodiment, the system 10 is further operable to (i) automatically select an internal-external rotation of the femoral prosthesis in the femur coordinate space that minimizes edge loading of the femoral and tibial prostheses and/or (ii) automatically review and/or update a selected internal-external rotation of the femoral prosthesis based on a currently planned tibia position (e.g., when the previously planned tibia position and the currently planned tibia position differ due to, for example, changes in the surgical plan). In the illustrative embodiment, the system 10 constrains flexion-extension and varus-valgus rotations of the femoral distal resection plane 108 (to keep it parallel to the tibia transverse resection plane 106 when the femur 24 and tibia 26 are positioned in full extension with the target HKA angle captured in block 206) but optimizes the internal-external rotation to make the femoral prosthesis as perpendicular as possible to the tibial prosthesis throughout the range of motion. It is contemplated that, in other embodiments, different implant designs might dictate different optimizations to minimize edge loading (e.g., seeking to best align the femoral and tibial prostheses at some predefined angle other than perpendicular). FIG. 15 illustrates one method 1500 for selecting the internal-external rotation of the femoral prosthesis based on an internal-external rotation of the tibial prosthesis (either planned or actual). This method 1500 may also be used to automatically review and/or update an existing internal-external rotation.

The method 1500 begins with block 1502, in which the system 10 defines a transverse vector in the tibia coordinate space that is representative of the internal-external rotation of the tibial prosthesis. The transverse vector lies in a transverse plane of the tibial prosthesis and is orthogonal to a vertical axis of the tibial prosthesis (i.e., the axis about which internal-external rotation is defined). For instance, the transverse vector may be defined along a sagittal axis of the tibial prosthesis or along a frontal axis of the tibial prosthesis (which are orthogonal to one another and to the vertical axis of the tibial prosthesis). In some cases, the internal-external rotation of the tibial prosthesis represented by the transverse vector may form part of a planned position of the tibial prosthesis defined in the surgical plan before the transverse and sagittal resections of the tibia have been performed. The planned position of the tibial prosthesis may have been selected by the system 10, or it may have been modified by a surgeon using the system 10. In other cases, the internal-external rotation of the tibial prosthesis represented by the transverse vector may form part of an actual position of the tibial prosthesis defined in the surgical plan after the transverse and sagittal resections of the tibia have been performed. In such cases, the system 10 is informed of the actual position of the tibial prosthesis by capturing measurements of the sagittal resected surface 104 and the transverse resected surface 106 of the tibia 26 in block 206.

After block 1502, the method 1500 proceeds to block 1504, in which the system 10 projects an instance of the transverse vector (defined in block 1502) from the tibia coordinate space into the femur coordinate space for each of the different poses of the knee joint for which the system 10 captured a spatial relationship between the femur coordinate space and the tibia coordinate space in block 206. For each such pose of the knee joint, the system 10 uses the corresponding spatial relationship between the two coordinate spaces to project the transverse vector from the tibia coordinate space into the femur coordinate space. In some embodiments, the projected vectors occupy three dimensions in the femur coordinate space. In other embodiments, when the transverse vectors are projected from the tibia coordinate space into the femur coordinate space, they are projected onto a two-dimensional transverse plane in the femur coordinate space (which is orthogonal to an anatomical axis of the femur in the femur coordinate space).

After block 1504, the method 1500 proceeds to block 1506, in which the system 10 combines the projected vectors from block 1504 to determine a composite vector in a transverse plane in the femur coordinate space. In embodiments in which the projected vectors occupy three dimensions in the femur coordinate space after block 1504, block 1506 involves computing an average vector from the plurality of projected vectors and then projecting that average vector onto the transverse plane in the femur coordinate space to produce the composite vector. In embodiments in which the projected vectors occupy the two-dimensional transverse plane in the femur coordinate space after block 1504, block 1506 involves computing an average vector from the plurality of projected vectors to produce the composite vector. In either embodiment, the composite vector produced in block 1506 lies in the transverse plane in the femur coordinate space and is representative of an average internal-external rotation of the tibial prosthesis, projected into the femur coordinate space.

After block 1506, the method 1500 proceeds to block 1508, in which the system 10 plans the internal-external rotation of the femoral prosthesis based on the composite vector produced in block 1506. For instance, where the transverse vector in the tibia coordinate space was defined along a sagittal axis of the tibial prosthesis (in block 1502), block 1508 may involve rotating the position of the femoral prosthesis in the femur coordinate space to align a sagittal axis of the femoral prosthesis with the composite vector. As another example, where the transverse vector in the tibia coordinate space was defined along a frontal axis of the tibial prosthesis (in block 1502), block 1508 may involve rotating the position of the femoral prosthesis in the femur coordinate space to align a frontal axis of the femoral prosthesis with the composite vector. As yet another example, where the transverse vector in the tibia coordinate space was defined along the frontal axis of the tibial prosthesis (in block 1502), block 1508 may involve rotating the position of the femoral prosthesis in the femur coordinate space to cause a sagittal axis of the femoral prosthesis to be orthogonal to both the composite vector and a mechanical axis of the femur.

In the illustrative embodiment, the system 10 is also operable to (i) automatically select a medial-lateral translation of the femoral prosthesis in the femur coordinate space that centers the femoral prosthesis over the tibial prosthesis and/or (ii) automatically review and/or update a selected medial-lateral translation of the femoral prosthesis due to, for example, changes in the surgical plan. Medial-lateral translation is the one degree of freedom of the position of the femoral prosthesis that is not constrained by the posterior resection plane 88, the distal resection plane 108, and the posterior chamfer resection plane 110 of the surgical plan (or vice versa). For medial-lateral translation, the system 10 will select a position for the femoral prosthesis that reduces or eliminates overhang for as much of the range of motion as possible. FIG. 16 illustrates one method 1600 for selecting and/or automatically reviewing and/or updating the medial-lateral translation of the femoral prosthesis based on predicated contact locations with the tibial prosthesis (for either planned or actual position).

The method 1600 begins with block 1602, in which the system 10 predicts a contact location (in the femur coordinate space) between femoral prosthesis and the tibial prosthesis for each of the different poses of the knee joint for which the system 10 captured a spatial relationship between the femur coordinate space and the tibia coordinate space in block 206. For each such pose of the knee joint, the system 10 uses the corresponding spatial relationship between the two coordinate spaces, together with the planned position of the femoral prosthesis in the femur coordinate space and the planned (or actual) position of the tibial prosthesis in the tibia coordinate space, to predict the contact location. In some embodiments, the contact location is a multi-dimensional contact area defined relative to the femur coordinate space. In other embodiments, the contact location is a point in the femur coordinate space that approximates the contact between the femoral and tibial prostheses (e.g., a centroid of a contact area).

In some cases, the position of the tibial prosthesis used to predict the contact locations is defined in the surgical plan before the transverse and sagittal resections of the tibia have been performed. The planned position of the tibial prosthesis may have been selected by the system 10, or it may have been modified by a surgeon using the system 10. In other cases, the position of the tibial prosthesis used to predict the contact locations is defined in the surgical plan after the transverse and sagittal resections of the tibia have been performed. In such cases, the system 10 is informed of the actual position of the tibial prosthesis by capturing measurements of the sagittal resected surface 104 and the transverse resected surface 106 of the tibia 26 in block 206.

After block 1602, the method 1600 proceeds to block 1604, in which the system 10 combines a medial-lateral aspect of each of the predicted contact locations from block 1602 to determine a composite location in the femur coordinate space. Block 1604 only considers the medial-lateral coordinate(s) of the contact locations predicted in block 1602. Block 1604 may involve computing an average of these medial-lateral aspects of the predicted contact locations from block 1602 to produce the composite location. In the illustrative embodiment, the composite location is a point that represents a medial-lateral center of all of the positions of the tibial prosthesis over the range of motion. After block 1604, the method 1600 proceeds to block 1606, in which the system 10 plans the medial-lateral translation of the femoral prosthesis based on the composite location. In the illustrative embodiment, block 1606 involves centering a mediolateral dimension of the femoral prosthesis on the composite location.

In the illustrative embodiment, the system 10 is further operable to automatically adjust certain surgical parameters of the surgical plan to achieve (to the extent possible) the target HKA angle set by the surgeon during block 206. As discussed above, the system 10 captures a spatial relationship between the femur coordinate space and the tibia coordinate space while the surgeon positions the femur 24 and the tibia 26 in full extension with the target HKA angle during block 206. This spatial relationship between the two coordinate spaces is used to adjust several surgical parameters of the surgical plan in the method 1700, which is illustrated in FIG. 17.

The method 1700 begins with block 1702, in which the system 10 calculates a planned gap distance between the femoral prosthesis and the tibial prosthesis based on the spatial relationship discussed above and the planned values for the position of the transverse resection plane 106 in the tibia coordinate space, a vertical dimension of the tibial prosthesis, the position of the distal resection plane 108 in the femur coordinate space, and a vertical dimension of the femoral prosthesis. As discussed above, the system 10 constrains transverse resection plane 106 and the distal resection plane 108 to remain parallel to one another while the two coordinate spaces have the spatial relationship of the target HKA angle. In the illustrative embodiment, block 1702 involves the system 10 calculating a distance between the transverse resection plane 106 and the distal resection plane 108 and subtracting either the vertical dimension of just the femoral prosthesis or the vertical dimensions of both prostheses (depending on the convention being used) to calculate the planned gap distance. As discussed above, it is also contemplated that some embodiments of the present disclosure may utilize non-planar resections of the femur 24 and/or tibia 26. In such embodiments, a closest distance between the planned resections can be used in place of the distance between the planes 106, 108 in method 1700.

After block 1702, the method 1700 proceeds to block 1704, in which the planned gap distance just calculated in block 1702 is compared to a threshold. Where the system 10 uses the vertical dimension of the tibial prosthesis when calculating the planned gap distance in block 1702, the threshold in block 1704 may be zero. Where the system 10 does not use the vertical dimension of the tibial prosthesis when calculating the planned gap distance in block 1702, the threshold in block 1704 may be a positive number, such as 7 mm or 8 mm, by way of example. In either case, the threshold used may also account for the resolution of the system 10. If the planned gap distance is closest to the threshold in block 1704 or does not exceed the threshold in block 1704, this predicts contact between the femoral prosthesis and the tibial prosthesis at the target HKA angle, and the method 1700 ends.

However, if the planned gap distance is not closest to the threshold in block 1704, the method may proceed to clock 1706. Alternatively, if the planned gap distance does exceed the threshold in block 1704, the method 1700 instead proceeds to block 1706. In block 1706, the system 10 updates at least one surgical parameter of the surgical plan. In the illustrative embodiment, on this first pass through block 1706, the system 10 implements block 1708, if possible. In block 1708, if a position of the transverse resection plane 106 is not fixed (i.e., the transverse resection of the tibia 26 has not yet been performed) and is not at a minimum allowable depth (e.g., 2 mm), the system 10 will shift the transverse resection plane 106 (or otherwise adjust the planned position of tibia prosthesis) proximally to attempt to close the gap distance. As such, block 1708 may involve shifting the transverse resection plane 106 proximally in the tibia coordinate space by the lesser of two differences: a difference between the planned gap distance and the threshold, and a difference between a current depth of the transverse resection plane 106 and the minimum allowable depth.

After block 1708 is performed, the method 1700 loops back to block 1702 in which the planned gap distance is calculated in the same manner describe above. After block 1702, the method 1700 proceeds again to block 1704, in which the planned gap distance just calculated in block 1702 is compared to the threshold. If the planned gap distance is closest to the threshold in block 1704, the method 1700 may end. Alternatively if the planned gap distance does not exceed the threshold in block 1704, this predicts contact between the femoral prosthesis and the tibial prosthesis at the target HKA angle, and the method 1700 ends.

However, if the planned gap distance does exceed the threshold in block 1704, the method 1700 may instead proceed to block 1706 in which the system 10 may update at least one surgical parameter of the surgical plan. In the illustrative embodiment, on this second pass through block 1706 (or whenever the adjustment of block 1708 is no longer possible), the system 10 implements block 1710, if possible. In block 1710, if a position of the distal resection plane 108 is not fixed (i.e., the distal resection of the femur 24 has not yet been performed) and is not at a minimum allowable depth (e.g., 4.5 mm), the system 10 will shift the distal resection plane 108 (or otherwise adjust the planned position of femoral prosthesis) distally to attempt to close the gap distance. As such, block 1710 may involve shifting the distal resection plane 108 distally in the femur coordinate space by the lesser of two differences: a difference between the planned gap distance and the threshold, and a difference between a current depth of the distal resection plane 108 and the minimum allowable depth.

After block 1710 is performed, the method 1700 loops back to block 1702 in which the planned gap distance is calculated in the same manner describe above. After block 1702, the method 1700 proceeds again to block 1704, in which the planned gap distance just calculated in block 1702 is compared to the threshold. If the planned gap distance does not exceed the threshold in block 1704, this predicts contact between the femoral prosthesis and the tibial prosthesis at the target HKA angle, and the method 1700 ends.

However, if the planned gap distance does exceed the threshold in block 1704, the method 1700 instead proceeds to block 1706 in which the system 10 updates at least one surgical parameter of the surgical plan. In the illustrative embodiment, on this third pass through block 1706 (or whenever the adjustments of block 1708 and block 1710 no longer possible), the system 10 implements block 1712, if possible. In block 1712, if a vertical dimension of the tibial prosthesis is not at a maximum possible value, the system 10 will select a new tibial prosthesis with a larger vertical dimension that minimizes that planned gap distance. In the illustrative embodiment, the tibial prosthesis comprises a tibial tray and a bearing insert, and block 1712 involves selecting a new bearing insert for use with the previously selected tibial tray.

While the disclosure has been illustrated and described in detail in the drawings and foregoing description, such an illustration and description is to be considered as exemplary and not restrictive in character, it being understood that only illustrative embodiments have been shown and described and that all changes and modifications that come within the spirit of the disclosure are desired to be protected.

There are a plurality of advantages of the present disclosure arising from the various features of the apparatus, system, and method described herein. It will be noted that alternative embodiments of the apparatus, system, and method of the present disclosure may not include all of the features described yet still benefit from at least some of the advantages of such features. Those of ordinary skill in the art may readily devise their own implementations of the apparatus, system, and method that incorporate one or more of the features of the present invention and fall within the spirit and scope of the present disclosure.

1. A method for an orthopaedic surgical procedure, the method comprising:
capturing, using a surgical navigation system, a plurality of measurements of a knee joint of a patient, wherein the plurality of measurements comprises (i) one or more measurements on an operative side of a femur of the patient relative to a femur coordinate space, (ii) one or more measurements on an operative side of a tibia of the patient relative to a tibia coordinate space, and (iii) a spatial relationship between the femur coordinate space and the tibia coordinate space at each of a plurality of different poses of the knee joint; and
developing, using the surgical navigation system, a surgical plan for the orthopaedic surgical procedure based on the plurality of measurements, wherein the surgical plan comprises (i) a position of a tibial prosthesis in the tibia coordinate space, including an internal-external rotation of the tibial prosthesis and (ii) a position of a femoral prosthesis in the femur coordinate space, including an internal-external rotation of the femoral prosthesis, and wherein developing the surgical plan comprises:
defining a transverse vector in the tibia coordinate space that is representative of the internal-external rotation of the tibial prosthesis;
for each of the plurality of different poses of the knee joint, using the corresponding spatial relationship between the femur coordinate space and the tibia coordinate space to project the transverse vector from the tibia coordinate space into the femur coordinate space;
combining the plurality of projected vectors to determine a composite vector in a transverse plane in the femur coordinate space; and
planning the internal-external rotation of the femoral prosthesis based on the composite vector.

2. The method of claim 1, wherein the position of the tibial prosthesis, including the internal-external rotation of the tibial prosthesis used when defining the transverse vector, is a planned position of the tibial prosthesis defined in the surgical plan before performing transverse and sagittal resections of the tibia during the orthopaedic surgical procedure.

3. The method of claim 2, wherein the planned position of the tibial prosthesis, including the internal-external rotation of the tibial prosthesis used when defining the transverse vector, has been modified by a surgeon from a default position set by the surgical navigation system.

4. The method of claim 1, wherein the position of the tibial prosthesis, including the internal-external rotation of the tibial prosthesis used when defining the transverse vector, is an actual position of the tibial prosthesis defined in the surgical plan after performing a transverse resection of the tibia and a sagittal resection of the tibia during the orthopaedic surgical procedure.

5. The method of claim 4, wherein capturing the one or more measurements on the operative side of the tibia comprises capturing at least one measurement on at least one of (i) a transverse resected surface created by the transverse resection of the tibia and (ii) a sagittal resected surface created by the sagittal resection of the tibia.

6. The method of claim 1, wherein the transverse vector in the tibia coordinate space is defined along a sagittal axis of the tibial prosthesis.

7. The method of claim 6, wherein planning the internal-external rotation of the femoral prosthesis comprises rotating the position of the femoral prosthesis in the femur coordinate space to align a sagittal axis of the femoral prosthesis with the composite vector.

8. The method of claim 1, wherein the transverse vector in the tibia coordinate space is defined along a frontal axis of the tibial prosthesis.

9. The method of claim 8, wherein planning the internal-external rotation of the femoral prosthesis comprises rotating the position of the femoral prosthesis in the femur coordinate space to align a frontal axis of the femoral prosthesis with the composite vector.

10. The method of claim 1, wherein combining the plurality of projected vectors comprises:
computing an average vector from the plurality of projected vectors; and
projecting the average vector onto the transverse plane to produce the composite vector.

11. The method of claim 1, wherein:
projecting the transverse vector from the tibia coordinate space into the femur coordinate space comprises projecting the transverse vector onto a transverse plane in the femur coordinate space; and
combining the plurality of projected vectors comprises computing an average vector from the plurality of projected vectors to produce the composite vector.

12. A method for an orthopaedic surgical procedure, the method comprising:
capturing, using a surgical navigation system, a plurality of measurements of a knee joint of a patient, wherein the plurality of measurements comprises (i) one or more measurements on an operative side of a femur of the patient relative to a femur coordinate space, (ii) one or more measurements on an operative side of a tibia of the patient relative to a tibia coordinate space, and (iii) a spatial relationship between the femur coordinate space and the tibia coordinate space at each of a plurality of different poses of the knee joint; and
developing, using the surgical navigation system, a surgical plan for the orthopaedic surgical procedure based on the plurality of measurements, wherein the surgical plan comprises (i) a position of a tibial prosthesis in the tibia coordinate space and (ii) a position of a femoral prosthesis in the femur coordinate space, including a medial-lateral translation of the femoral prosthesis, and wherein developing the surgical plan comprises:
for each of the plurality of different poses of the knee joint, using the corresponding spatial relationship between the femur coordinate space and the tibia coordinate space, together with the position of the femoral prosthesis in the femur coordinate space and the position of the tibial prosthesis in the tibia coordinate space, to predict a contact location between the femoral prosthesis and the tibial prosthesis in the femur coordinate space;
combining a medial-lateral aspect of each of the plurality of predicted contact locations to determine a composite location in the femur coordinate space; and
planning the medial-lateral translation of the femoral prosthesis based on the composite location.

13. The method of claim 12, wherein the position of the tibial prosthesis used to determine the plurality of predicted contact locations is a planned position of the tibial prosthesis defined in the surgical plan before performing transverse and sagittal resections of the tibia during the orthopaedic surgical procedure.

14. The method of claim 13, wherein the planned position of the tibial prosthesis used to determine the plurality of predicted contact locations has been modified by a surgeon from a default position set by the surgical navigation system.

15. The method of claim 12, wherein the position of the tibial prosthesis used to determine the plurality of predicted contact locations is an actual position of the tibial prosthesis defined in the surgical plan after performing a transverse resection of the tibia and a sagittal resection of the tibia during the orthopaedic surgical procedure.

16. The method of claim 15, wherein capturing the one or more measurements on the operative side of the tibia comprises capturing at least one measurement on at least one of (i) a transverse resected surface created by the transverse resection of the tibia and (ii) a sagittal resected surface created by the sagittal resection of the tibia.

17. The method of claim 12, wherein:
combining the medial-lateral aspect of each of the plurality of predicted contact locations comprises computing an average of the medial-lateral aspects to produce the composite location; and
planning the medial-lateral translation comprises centering a mediolateral dimension of the femoral prosthesis on the composite location.

18. The method of claim 12, wherein the plurality of different poses of the knee joint comprises a first pose in which the femur and the tibia are in full extension and a second pose in which the femur and the tibia are flexed at 90 degrees.

19. The method of claim 18, wherein the plurality of different poses of the knee joint further comprises additional poses in a range of motion between the first pose and the second pose.

20. The method of claim 19, wherein the additional poses are spaced at regular intervals of flexion between the first pose and the second pose.

21. The method of claim 12, wherein developing the surgical plan further comprises determining a plurality of planes in the femur coordinate space for a plurality of resections of the femur based on the surgical plan's position of the femoral prosthesis.

22. A method for an orthopaedic surgical procedure, the method comprising:
capturing, using a surgical navigation system, a plurality of measurements of a femur of a patient relative to a femur coordinate space, wherein the plurality of measurements comprises (i) a most posterior point on an operative side of the femur and (ii) an anterior sizing point on the operative side of the femur; and
developing, using the surgical navigation system, a surgical plan for the orthopaedic surgical procedure based on the plurality of measurements, wherein the surgical plan comprises (i) a plurality of planes in the femur coordinate space for a plurality of resections of the femur and (ii) a size of a femoral prosthesis to be implanted on resected surfaces created by the plurality of resections of the femur, and wherein developing the surgical plan comprises selecting the size of the femoral prosthesis from among a plurality of possible sizes for the femoral prosthesis based on the most posterior point and the anterior sizing point.

23. The method of claim 22, wherein capturing the anterior sizing point on the operative side of the femur comprises:
placing the femur and a tibia of the patient into full extension; and
touching a tip of a pointer instrument of the surgical navigation system to a point on the operative side of the femur that is directly proximal of a most anterior point of an anticipated transverse resection to be performed on the tibia.

24. The method of claim 22, wherein capturing the anterior sizing point on the operative side of the femur comprises:
placing the femur and a tibia of the patient into full extension; and
touching a tip of a pointer instrument of the surgical navigation system to a point on the operative side of the femur that is directly proximal of a most anterior point of an actual transverse resection performed on the tibia.

25. The method of claim 22, wherein selecting the size of the femoral prosthesis comprises:
calculating a transverse distance in the femur coordinate space between (i) the most posterior point on the operative side of the femur and (ii) the anterior sizing point on the operative side of the femur; and
selecting, from among the plurality of possible sizes for the femoral prosthesis, the possible size with the largest anteroposterior dimension that does not exceed the transverse distance by more than one-half of a resolution of the surgical navigation system.

26. The method of claim 22, wherein selecting the size of the femoral prosthesis comprises:
aligning, in the femur coordinate space, a digital model of each of the plurality of possible sizes for the femoral prosthesis to the most posterior point on the operative side of the femur; and
selecting, from among the plurality of possible sizes for the femoral prosthesis, the possible size corresponding to the aligned digital model with a smallest distance between (i) the anterior sizing point on the operative side of the femur and (ii) a most anterior point of the aligned digital model.

27. The method of claim 22, further comprising performing, using the surgical navigation system, the plurality of resections of the femur according to the surgical plan.

28. The method of claim 27, wherein performing the plurality of resections of the femur according to the surgical plan comprises operating a robotic assisted surgery device in communication with the surgical navigation system, and wherein, for each of the plurality of resections of the femur, the robotic assisted surgery device constrains movement of a surgical saw blade to a corresponding plane of the plurality of planes of the surgical plan.

29. A method for an orthopaedic surgical procedure, the method comprising:
capturing, using a surgical navigation system, a plurality of measurements of a knee joint of a patient, wherein the plurality of measurements comprises (i) one or more measurements on an operative side of a femur of the patient relative to a femur coordinate space, (ii) one or more measurements on an operative side of a tibia of the patient relative to a tibia coordinate space, and (iii) a spatial relationship between the femur coordinate space and the tibia coordinate space while a surgeon positions the femur and the tibia in full extension with a target hip-knee-ankle angle; and
developing, using the surgical navigation system, a surgical plan for the orthopaedic surgical procedure based on the plurality of measurements, wherein the surgical plan comprises (i) a transverse plane in the tibia coordinate space for a transverse resection of the tibia, (ii) a vertical dimension of a tibial prosthesis to be implanted on a transverse resected surface created by the transverse resection of the tibia, (iii) a distal plane in the femur coordinate space for a distal resection of the femur, and (iv) a vertical dimension of a femoral prosthesis to be implanted on a distal resected surface created by the distal resection of the femur, and wherein developing the surgical plan comprises:
calculating a first gap distance between the femoral prosthesis and the tibial prosthesis based on the transverse plane in the tibia coordinate space, the vertical dimension of the tibial prosthesis, the distal plane in the femur coordinate space, the vertical dimension of the femoral prosthesis, and the spatial relationship between the femur coordinate space and the tibia coordinate space; and
in response to the first gap distance being greater than one-half of a resolution of the surgical navigation system, updating the surgical plan by shifting the transverse plane proximally in the tibia coordinate space by the lesser of (i) the first gap distance and (ii) a current depth of the transverse resection of the tibia minus a minimum allowable value for the transverse resection of the tibia.

30. The method of claim 29, wherein developing the surgical plan further comprises:
after updating the surgical plan by shifting the transverse plane proximally in the tibia coordinate space, calculating a second gap distance between the femoral prosthesis and the tibial prosthesis based on the transverse plane in the tibia coordinate space, the vertical dimension of the tibial prosthesis, the distal plane in the femur coordinate space, the vertical dimension of the femoral prosthesis, and the spatial relationship between the femur coordinate space and the tibia coordinate space; and
in response to the second gap distance being greater than one-half of a resolution of the surgical navigation system, updating the surgical plan by shifting the distal plane distally in the femur coordinate space by the lesser of (i) the second gap distance and (ii) a current depth of the distal resection of the femur minus a minimum allowable value for the distal resection of the femur.

31. The method of claim 30, wherein developing the surgical plan further comprises:
after updating the surgical plan by shifting the distal plane distally in the femur coordinate space, calculating a third gap distance between the femoral prosthesis and the tibial prosthesis based on the transverse plane in the tibia coordinate space, the vertical dimension of the tibial prosthesis, the distal plane in the femur coordinate space, the vertical dimension of the femoral prosthesis, and the spatial relationship between the femur coordinate space and the tibia coordinate space; and
in response to the third gap distance being greater than one-half of a resolution of the surgical navigation system, updating the surgical plan by selecting a new tibial prosthesis with a larger vertical dimension that minimizes that third gap distance.

32. The method of claim 31, wherein the tibial prosthesis comprises a tibial tray and a bearing insert, and wherein selecting the new tibial prosthesis with the larger vertical dimension comprises selecting a new bearing insert for use with the tibial tray.

33. The method of claim 29, wherein the distal plane is parallel to the transverse plane when the femur coordinate space and the tibia coordinate space have the spatial relationship.

34. The method of claim 29, further comprising performing, using the surgical navigation system, the transverse resection of the tibia and the distal resection of the femur according to the surgical plan.

35. The method of claim 34, wherein performing the transverse resection of the tibia and the distal resection of the femur according to the surgical plan comprises operating a robotic assisted surgery device in communication with the surgical navigation system, wherein the robotic assisted surgery device constrains movement of a surgical saw blade to the transverse plane during the transverse resection of the tibia, and wherein the robotic assisted surgery device constrains movement of the surgical saw blade to the distal plane during the distal resection of the femur.

36. A method for an orthopaedic surgical procedure, the method comprising:
capturing, using a surgical navigation system, a plurality of measurements of a tibia of a patient relative to a tibia coordinate space, wherein the plurality of measurements comprises (i) a most collateral point on an operative side of the tibia and (ii) a tibial compartment border on the operative side of the tibia; and
developing, using the surgical navigation system, a surgical plan for the orthopaedic surgical procedure based on the plurality of measurements, wherein the surgical plan comprises (i) a plane in the tibia coordinate space for a transverse resection of the tibia and (ii) a size of a tibial tray of a tibial prosthesis to be implanted on a transverse resected surface created by the transverse resection of the tibia, and wherein developing the surgical plan comprises selecting the size of the tibial tray from among a plurality of possible sizes for the tibial tray based on the most collateral point and the tibial compartment border.

37. The method of claim 36, wherein capturing the tibial compartment border on the operative side of the tibia comprises aligning an elongated body of a pointer instrument of the surgical navigation system with the tibial compartment border, prior to performing the transverse resection of the tibia.

38. The method of claim 37, wherein capturing the most collateral point on the operative side of the tibia comprises touching a tip of the pointer instrument to a point in an anticipated plane of the transverse resection of the tibia, prior to performing the transverse resection.

39. The method of claim 36, wherein capturing the tibial compartment border on the operative side of the tibia comprises moving a tip of a pointer instrument of the surgical navigation system along a sagittal resected surface created by a sagittal resection of the tibia, after performing the transverse and sagittal resections of the tibia.

40. The method of claim 39, wherein capturing the most collateral point on the operative side of the tibia comprises touching the tip of the pointer instrument to a point in an actual plane of the transverse resection of the tibia, after performing the transverse resection of the tibia.

41. The method of claim 36, wherein selecting the size of the tibial tray comprises:
calculating a smallest distance in the tibia coordinate space between (i) the most collateral point on the operative side of the tibia and (ii) the tibial compartment border on the operative side of the tibia; and
selecting, from among the plurality of possible sizes for the tibial tray, the possible size with the largest mediolateral dimension that does not exceed the smallest distance by more than one-half of a resolution of the surgical navigation system.

42. The method of claim 36, wherein selecting the size of the tibial tray comprises:
aligning, in the tibia coordinate space, a digital model of each of the plurality of possible sizes for the tibial tray to the tibial compartment border on the operative side of the tibia; and
selecting, from among the plurality of possible sizes for the tibial tray, the possible size corresponding to the aligned digital model with a smallest distance between (i) the most collateral point on the operative side of the tibia and (ii) a most collateral point of the aligned digital model.

43. The method of claim 36, further comprising performing, using the surgical navigation system, the transverse resection of the tibia according to the surgical plan.

44. The method of claim 43, wherein performing the transverse resection of the tibia according to the surgical plan comprises operating a robotic assisted surgery device in communication with the surgical navigation system, and wherein the robotic assisted surgery device constrains movement of a surgical saw blade to the surgical plan's plane for the transverse resection of the tibia.
